(19) United States
(12) Patent Application Publication
(10) Pub. No.: US 2026/0364222 A1
(43) Pub. Date: Nov. 19, 2026

(54) GRAVITY-FED VOLATILE LIQUID AIR FRESHENING SYSTEMS

(71) Applicant: Fresh Products, Inc., Perrysburg, OH (US)

(72) Inventors: Douglas S. Brown, Toledo, OH (US); Jeffrey A. Smith, Perrysburg, OH (US)

(21) Appl. No.: 19/673,144

(22) Filed: May 11, 2026

Related U.S. Application Data
(60) Provisional application No. 63/804,921, filed on May 13, 2025.

(51) Int. Cl. A61L 9/12; F24F 8/50

(52) U.S. Cl. CPC A61L 9/127; F24F 8/50; A61L 2209/133

(57) ABSTRACT

An air freshener includes a reservoir body that at least partially defines a liquid chamber; a volatile liquid positioned within the liquid chamber; a first wick that covers an opening of the reservoir body; a vessel that at least partially defines an air chamber positioned substantially around the liquid chamber; and a lid coupled to a first end of the vessel and positioned such that the liquid chamber is fluidly sealed from an external environment except through the first wick, wherein the air chamber is vented to the external environment.

### CROSS-REFERENCE TO RELATED APPLICATIONS

This application claims the benefit of U.S. Provisional Application No. 63/804,921, titled GRAVITY-FED VOLATILE LIQUID AIR FRESHENING SYSTEMS, filed May 13, 2025, the entirety of which is hereby incorporated by reference herein.

### BACKGROUND

### Field

This disclosure relates generally to air fresheners, and more specifically to air fresheners that utilize volatile liquids.

### SUMMARY

Disclosed herein are various embodiments of air fresheners that utilize a volatile liquid, such as a formulation of a plurality of volatile chemicals, to release fragrance into an environment. Various embodiments are configured in a passive arrangement that utilizes gravity to feed the volatile liquid through a wick with a fragrance container in an orientation that positions the wick below the volatile liquid, such as in an inverted orientation. Various air fresheners disclosed herein are capable of outputting a relatively consistent amount of fragrance over an extended period of time. Additionally, various embodiments of air fresheners disclosed herein include features to reduce leakage of the volatile liquid and/or to reduce the effects of an external environment on the air freshener.

According to some embodiments, an air freshener comprises: a reservoir body comprising a first wall that at least partially defines a liquid chamber; a volatile liquid positioned within the liquid chamber of the reservoir body; a first wick that covers an opening of the reservoir body, wherein the first wick comprises a sheet of wicking material; a vessel comprising a second wall that at least partially defines an air chamber, wherein the first wall of the reservoir body is positioned substantially radially inward of the second wall, such that an exterior surface of the first wall is in contact with air positioned within the air chamber, and an interior surface of the first wall is in contact with the volatile liquid positioned within the liquid chamber; and a lid coupled to a first end of the vessel and positioned such that the liquid chamber is fluidly sealed from an external environment except through the first wick, wherein the air chamber is vented to the external environment.

In some embodiments, the air freshener further comprises a holder that positions a second wick below and spaced apart from the first wick, with the liquid chamber positioned above the first wick. In some embodiments, the air chamber is vented to the external environment at least through a flow path that passes through an area between the vessel and the lid. In some embodiments, the air freshener further comprises a seal between the reservoir body and the lid that restricts fluid communication from an external side of the first wick to the external environment through the area between the vessel and the lid. In some embodiments, the air chamber is vented to the external environment through at least one opening in at least one of the second wall of the vessel or a base of the vessel. In some embodiments, the first wall of the reservoir body is positioned such that at least 75% of the air positioned within the air chamber is positioned within an annular space between the first wall of the reservoir body and the second wall of the vessel. In some embodiments, a volume of the liquid chamber is at least 25% of a volume of the air chamber. In some embodiments, a volume of the liquid chamber is at least 75% of a volume of the air chamber. In some embodiments, the air freshener further comprises a cover positioned to prevent the first wick from being exposed to the external environment until the cover is moved away from a closed position. In some embodiments, the cover is part of the lid. In some embodiments, a volume of the liquid chamber is at least partially defined by the first wick, wherein the first wick comprises a relaxed state and a deformed state, with the deformed state corresponding to a smaller liquid chamber volume than the relaxed state, and wherein the cover comprises an inwardly protruding portion positioned such that, with the cover in the closed position, the inwardly protruding portion of the cover forces the first wick into the deformed state. In some embodiments, the first wick comprises an outer portion that is compressed between the lid and the reservoir body, and an inner portion that is radially inward of the outer portion and that is positioned over the opening of the reservoir body, wherein the inner portion of the first wick deflects along an axial direction between the deformed state and the relaxed state, and wherein the outer portion of the first wick does not deflect along the axial direction between the deformed state and the relaxed state. In some embodiments, the wicking material is pulp-based wicking material.

According to some embodiments, an air freshener comprises: a reservoir body comprising a first wall that at least partially defines a liquid chamber; a volatile liquid positioned within the liquid chamber of the reservoir body; a first wick that covers an opening of the reservoir body, such that the liquid chamber is fluidly sealed from an external environment except through the first wick, wherein the first wick comprises a sheet of wicking material; and a second wall positioned around and spaced apart from the first wall of the reservoir body, forming an annular space between the first wall of the reservoir body and the second wall, wherein the annular space is in fluid communication with the external environment.

In some embodiments, the air freshener further comprises a second wick spaced apart from the first wick and positioned such that, with the volatile liquid positioned above the first wick, the second wick will be below the first wick. In some embodiments, the air freshener further comprises a holder that supports the first wick above the second wick. In some embodiments, the second wall is part of a vessel within which the reservoir body is substantially positioned. In some embodiments, the air freshener further comprises a cover positioned to prevent the first wick from being exposed to the external environment until the cover is moved away from a closed position. In some embodiments, the cover is part of a lid that maintains the reservoir body in a fixed position with respect to the second wall. In some embodiments, a volume of the liquid chamber is at least partially defined by the first wick, wherein the first wick comprises a relaxed state and a deformed state, with the deformed state corresponding to a smaller liquid chamber volume than the relaxed state, and wherein the cover comprises an inwardly protruding portion positioned such that, with the cover in the closed position, the inwardly protruding portion of the cover forces the first wick into the deformed state. In some embodiments, the first wick comprises an inner portion that is positioned over the opening of the reservoir body, and an outer portion that is positioned radially outward of the inner portion and the opening of the reservoir body, wherein the inner portion of the first wick deflects along an axial direction between the deformed state and the relaxed state, and wherein the outer portion of the first wick does not deflect along the axial direction between the deformed state and the relaxed state. In some embodiments, the wicking material is pulp-based wicking material.

According to some embodiments, an air freshener comprises: a reservoir body comprising a first wall that at least partially defines a liquid chamber; a volatile liquid positioned within the liquid chamber of the reservoir body; a first wick that covers an opening into the liquid chamber, such that the liquid chamber is fluidly sealed from an external environment except through the first wick, wherein the first wick comprises a sheet of wicking material; and a cover positioned to prevent the first wick from being exposed to the external environment until the cover is moved away from a closed position, wherein a volume of the liquid chamber is at least partially defined by the first wick, wherein the first wick comprises a relaxed state and a deformed state, with the deformed state corresponding to a smaller liquid chamber volume than the relaxed state, and wherein the cover comprises an inwardly protruding portion positioned such that, with the cover in the closed position, the inwardly protruding portion of the cover forces the first wick into the deformed state.

In some embodiments, the air freshener further comprises a lid, wherein the cover is part of the lid. In some embodiments, the first wick comprises an outer portion that is compressed between the lid and a surface beneath the outer portion, and an inner portion that is positioned over the opening, wherein the inner portion of the first wick deflects along an axial direction between the deformed state and the relaxed state, and wherein the outer portion of the first wick does not deflect along the axial direction between the deformed state and the relaxed state. In some embodiments, the wicking material is pulp-based wicking material. In some embodiments, the air freshener further comprises a vessel comprising a second wall that at least partially defines an air chamber, wherein the first wall of the reservoir body is positioned substantially radially inward of the second wall, such that an exterior surface of the first wall is in contact with air positioned within the air chamber and an interior surface of the first wall is in contact with the volatile liquid positioned within the liquid chamber, and wherein the air chamber is vented to the external environment. In some embodiments, the air freshener further comprises a lid that maintains the reservoir body in a fixed position with respect to the vessel. In some embodiments, the cover is part of the lid. In some embodiments, the air freshener further comprises a holder that positions a second wick below and spaced apart from the first wick, with the liquid chamber positioned above the first wick.

According to some embodiments, an air freshener comprises: a fragrance container; a volatile liquid positioned within a cavity of the fragrance container, wherein the volatile liquid is a chemical blend, at least 90% by volume of which consists of a plurality of chemicals all having a vapor pressure within a 1mmHg range (i.e. a vapor pressure difference between a most volatile chemical of the plurality of chemicals and a least volatile chemical of the plurality of chemicals is no greater than 1mmHg); a first wick that covers an opening at an end of the fragrance container, such that the cavity of the fragrance container is fluidly sealed from an external environment except for through the first wick, wherein the first wick comprises a sheet of pulp-based wicking material, wherein an exposure surface area of the first wick is exposed to the external environment; a holder shaped to support the fragrance container in an orientation with the first wick facing downward, such that gravity will force the volatile liquid into contact with the first wick and cause the volatile liquid to evaporate through the first wick; and a second wick that comprises a sheet of pulp-based wicking material, wherein the second wick is supported by the holder spaced apart from the first wick in an orientation that positions the second wick to catch any liquid drops of the volatile liquid that fall unevaporated from the first wick.

In some embodiments, a ratio of the exposure surface area of the first wick to an average vapor pressure of the plurality of chemicals is within a range of 1500-5000, with the exposure surface area measured in mm² and the average vapor pressure measured in mmHg. In some embodiments, the plurality of chemicals all have a vapor pressure within 0.5mmHg. In some embodiments, at least 95% by volume of the chemical blend consists of a plurality of chemicals all having a vapor pressure within a 1mmHg range. In some embodiments, the plurality of chemicals comprises at least three chemicals that each produce a different aroma. In some embodiments, the plurality of chemicals comprises at least: a first chemical that produces an aroma classified as one of the following three types of notes: top note, medium note, or base note; and a second chemical that produces an aroma classified as one of the following three types of notes: top note, medium note, or base note, but classified differently than the first chemical. In some embodiments, the holder comprises an annularly shaped portion within which at least a portion of the fragrance container can be placed. In some embodiments, the holder further comprises a ledge extending inwardly from the annularly shaped portion, the ledge positioned to support the fragrance container.

According to some embodiments, an air freshener comprises: a fragrance container; a volatile liquid positioned within a cavity of the fragrance container; a first wick that covers an opening at an end of the fragrance container, such that the cavity of the fragrance container is fluidly sealed from an external environment except for through the first wick; a support structure shaped to support the fragrance container in an orientation with the first wick facing downward, such that gravity will force the volatile liquid into contact with the first wick and cause the volatile liquid to evaporate through the first wick; and a second wick that is supported by the support structure spaced apart from the first wick in an orientation that positions the second wick to catch any liquid drops of the volatile liquid that fall unevaporated from the first wick.

In some embodiments, the support structure is part of the fragrance container. In some embodiments, the support structure is part of a holder to which the fragrance container is removably coupleable. In some embodiments, the holder comprises one or more apertures for fastening the holder to a wall with one or more fasteners. In some embodiments, the holder comprises an annularly shaped portion within which at least a portion of the fragrance container can be placed. In some embodiments, the support structure comprises a ledge extending inwardly from the annularly shaped portion of the holder, the ledge positioned to support the fragrance container. In some embodiments, the support structure comprises one or more legs extending below the first wick to a drip collection member that supports the second wick, wherein the one or more legs are positioned to create a space between the first wick and the second wick that is exposed to the atmosphere. In some embodiments, the first wick comprises a sheet of pulp-based wicking material. In some embodiments, the second wick comprises a sheet of pulp-based wicking material. In some embodiments, the volatile liquid is a chemical blend, at least 90% by volume of which consists of a plurality of chemicals all having a vapor pressure within a 1mmHg range (i.e. a vapor pressure difference between a most volatile chemical of the plurality of chemicals and a least volatile chemical of the plurality of chemicals is no greater than 1mmHg). In some embodiments, the plurality of chemicals comprises at least three chemicals that each produce a different aroma. In some embodiments, the plurality of chemicals comprises at least: a first chemical that produces an aroma classified as one of the following three types of notes: top note, medium note, or base note; and a second chemical that produces an aroma classified as one of the following three types of notes: top note, medium note, or base note, but classified differently than the first chemical. In some embodiments, an exposure surface area of the first wick is exposed to the external environment, and wherein a ratio of the exposure surface area of the first wick to an average vapor pressure of the plurality of chemicals is within a range of 1500-5000, with the exposure surface area measured in mm² and the average vapor pressure measured in mmHg.

According to some embodiments, an air freshener comprises: a fragrance container having a cavity for positioning therein of a volatile liquid; one or more wicks positioned such that the volatile liquid will be fluidly sealed from an external environment other than through the one or more wicks; and wherein the one or more wicks are positioned such that, with the fragrance container in an orientation with an exposed area of the one or more wicks facing downward, the volatile liquid will be forced into contact with the one or more wicks by gravity, and the volatile liquid will evaporate through the exposed area of the one or more wicks to the external environment.

In some embodiments, the air freshener further comprises a drip collection member spaced apart from the one or more wicks and sized and positioned such that, with the fragrance container in the orientation with the exposed area of the one or more wicks facing downward, any drops of the volatile liquid that pass through the one or more wicks without evaporating will be caught by the drip collection member. In some embodiments, the air freshener further comprises a support structure that supports the drip collection member and creates a space between the one or more wicks and the drip collection member that is exposed to the external environment. In some embodiments, the support structure is part of the fragrance container. In some embodiments, the support structure is part of a holder to which the fragrance container is removably coupleable. In some embodiments, the drip collection member comprises an additional wick. In some embodiments, the drip collection member comprises an absorbent mat. In some embodiments, the one or more wicks each comprise a sheet of pulp-based wick material. In some embodiments, the air freshener further comprises a holder configured to support the fragrance container in the orientation with the one or more wicks facing downward, wherein the holder comprises the drip collection member. In some embodiments, the holder comprises one or more apertures for fastening the holder to a wall. In some embodiments, the holder comprises an annularly shaped portion within which at least a portion of the fragrance container can be placed. In some embodiments, the holder further comprises a ledge extending inwardly from the annularly shaped portion of the holder, the ledge positioned to support the fragrance container. In some embodiments, the air freshener further comprises the volatile liquid positioned in the cavity of the fragrance container, wherein the volatile liquid is a chemical formulation, at least 80% by volume of which consists of a plurality of chemicals all having a vapor pressure within a 3 mmHg range (i.e. a vapor pressure difference between a most volatile chemical of the plurality of chemicals and a least volatile chemical of the plurality of chemicals is no greater than 3mmHg). In some embodiments, the air freshener further comprises the volatile liquid positioned in the cavity of the fragrance container, wherein the volatile liquid is a chemical formulation, at least 90% by volume of which consists of a plurality of chemicals all having a vapor pressure within a 2mmHg range (i.e. a vapor pressure difference between a most volatile chemical of the plurality of chemicals and a least volatile chemical of the plurality of chemicals is no greater than 2mmHg). In some embodiments, the air freshener further comprises the volatile liquid positioned in the cavity of the fragrance container, wherein the volatile liquid is a chemical formulation, at least 90% by volume of which consists of a plurality of chemicals all having a vapor pressure within a 1mmHg range (i.e. a vapor pressure difference between a most volatile chemical of the plurality of chemicals and a least volatile chemical of the plurality of chemicals is no greater than 1mmHg). In some embodiments, the plurality of chemicals comprises at least three chemicals that each produce a different aroma. In some embodiments, the plurality of chemicals comprises at least: a first chemical that produces an aroma classified as one of the following three types of notes: top note, medium note, or base note; and a second chemical that produces an aroma classified as one of the following three types of notes: top note, medium note, or base note, but classified differently than the first chemical. In some embodiments, the plurality of chemicals comprises at least: a first chemical that produces an aroma classified as top note; a second chemical that produces an aroma classified as a medium note; and a third chemical that produces an aroma classified as a base note. In some embodiments, the plurality of chemicals comprises at least: a first chemical that produces an aroma selected from the following group of aromas: citrus, lemongrass, lemon, orange, bergamot, rose, lavender, anise, and basil; a second chemical that produces an aroma selected from the following group of aromas: iris, orchid, geranium, jasmine, neroli, ylang-ylang, black pepper, cardamom, pine, and cinnamon; and a third chemical that produces an aroma selected from the following group of aromas: moss, patchouli, musk, amber, vanilla, sandalwood, and cedarwood.

For purposes of this summary, certain aspects, advantages, and novel features of the inventions are described herein. It is to be understood that not necessarily all such advantages may be achieved in accordance with any particular embodiment of the inventions. Thus, for example, those skilled in the art will recognize that the inventions may be embodied or carried out in a manner that achieves one advantage or group of advantages as taught herein without necessarily achieving other advantages as may be taught or suggested herein.

### BRIEF DESCRIPTION OF THE DRAWINGS

The foregoing and other features, aspects, and advantages of the present disclosure are described in detail below with reference to the drawings of various embodiments, which are intended to illustrate and not to limit the disclosure. The features of some embodiments of the present disclosure, which are believed to be novel, will be more fully disclosed in the following detailed description. The following detailed description may best be understood by reference to the accompanying drawings wherein the same numbers in different drawings represents the same parts. All drawings are schematic and are not intended to show any dimension to scale. The drawings comprise the following figures in which:

FIG. 1A is a perspective view of an embodiment of an air freshener that includes a fragrance container supported in an inverted orientation by a holder.

FIG. 1B is another perspective view of the air freshener of FIG. 1A.

FIG. 1C is an exploded view of the air freshener of FIG. 1A.

FIG. 1D is a perspective view of the fragrance container of the air freshener of FIG. 1A.

FIG. 1E is a perspective view of the fragrance container of the air freshener of FIG. 1A, with a cover installed.

FIG. 1F is a perspective view of the holder of the air freshener of FIG. 1A.

FIG. 2 illustrates the air freshener of FIG. 1A in an example environment.

FIG. 3A is a schematic cross-section view of the fragrance container of the air freshener of FIG. 1A.

FIG. 3B is a schematic cross-section view of the holder of the air freshener of FIG. 1A.

FIG. 3C is a schematic cross-section view of the fragrance container of the air freshener of FIG. 1A, inverted and supported by the holder of the air freshener of FIG. 1A.

FIG. 4 is an example evaporation chart illustrating evaporation characteristics over time.

FIG. 5 is a schematic perspective view of another embodiment of an air freshener.

FIG. 6A is a schematic top view of a lid of the fragrance container of the air freshener of FIG. 1A.

FIGS. 6B and 6C are schematic top views of alternative embodiments of fragrance container lids.

FIG. 7 is a schematic cross-section view of another embodiment of a fragrance container.

FIG. 8A is a perspective view of another embodiment of a fragrance container.

FIG. 8B is a top view of the fragrance container of FIG. 8A.

FIG. 8C is a cross-section view of the fragrance container of FIG. 8A.

FIG. 8D is another cross-section view of the fragrance container of FIG. 8A.

FIG. 8E is an enlarged view of a portion of the cross-section view of FIG. 8D.

FIG. 9 is a cross-section view of an embodiment of an air freshener that includes the fragrance container of FIG. 8A.

FIG. 10A is a perspective view of a reservoir body of the fragrance container of FIG. 8A.

FIG. 10B is a perspective view of a vessel of the fragrance container of FIG. 8A.

FIG. 10C is a perspective view of a lid of the fragrance container of FIG. 8A.

FIG. 11 is a cross-section view of another embodiment of a fragrance container.

### DETAILED DESCRIPTION

Although several embodiments, examples, and illustrations are disclosed below, it will be understood by those of ordinary skill in the art that the inventions described herein extend beyond the specifically disclosed embodiments, examples, and illustrations and include other uses of the inventions and obvious modifications and equivalents thereof. Embodiments of the inventions are described with reference to the accompanying figures, wherein like numerals refer to like elements throughout. These drawings are considered to be a part of the entire description of some embodiments of the inventions. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

Passive air fresheners can be desirable because, for example, they do not require a power source. One challenge with passive air fresheners, however—particularly with passive air fresheners that utilize volatile chemicals evaporated through a wick—is that they typically are unable to release a consistent amount of fragrance over a relatively long period of time. Instead, passive air fresheners will typically release a relatively high amount of fragrance at the beginning, and relatively quickly drop off to a much lower amount of fragrance release.

Disclosed herein are various embodiments of air fresheners that utilize a volatile liquid, such as a formulation or blend of a plurality of volatile chemicals, to release fragrance into an environment. Various embodiments are configured in a passive arrangement that utilizes gravity to feed the volatile liquid through a sheet-style wick with a fragrance container in an orientation that positions the wick below the volatile liquid, such as in an inverted orientation. Various embodiments of air fresheners disclosed herein can have a number of benefits over prior air freshener designs. For example, the inverted gravity-fed passive air freshener designs disclosed herein can output a relatively consistent amount of fragrance over an extended period of time, without requiring a power source, a heating element, an atomizer, or the like. For example, some embodiments disclosed herein are capable of releasing a relatively consistent amount of fragrance for 60 days or more, and various embodiments can be modified or adjusted to last for varying numbers of days, including more or less than 60 days.

Various embodiments disclosed herein utilize a fragrance container that contains a volume of volatile liquid and that includes one or more flat, sheet, and/or disc-shaped wicks at an end of the container (such as at a top of the container). The one or more wicks may comprise, for example, a sheet, a disc, a flat sheet, a flat disc, another shape having a relatively high width-to-thickness ratio and oriented such that the primary method of transferring fragrance to the environment comprises transferring liquid through the thickness of the wick from an interior or wetted surface to an exterior, evaporative, or exposed surface, and/or the like. The fragrance container may include a cover over the wick that seals the wick and the volatile liquid from an external environment until the air freshener is ready to be put into use. When the air freshener is to be put into use, a user may remove the cover, exposing the one or more flat, sheet, and/or disc-shaped wicks to the external environment. The user can then turn over or invert the container, such that the wick in the top of the container is now facing downward and gravity pulls the volatile liquid blend into contact with the wick (e.g., with the interior or wetted surface of the wick). Once the wick is loaded up with the volatile liquid (e.g., the volatile liquid saturates the wick by flowing from the interior or wetted surface to the exterior, evaporative, or exposed surface), as long as the fragrance container remains inverted, the volatile liquid can tend to evaporate into the environment through the wick at a relatively consistent rate, until the fragrance container runs out of volatile liquid. Alternatively, in some embodiments, the wick may already be positioned below the volatile liquid blend before the cover is removed, and the user may not need to invert the container. In such a configuration, the wick may already be loaded up with the volatile liquid before the cover is removed.

Additionally, in some embodiments, the cover that is over the wick and seals the wick and the volatile liquid from the external environment until the air freshener is ready to be put into use includes one or more features that press on or otherwise deform the wick while the cover is in place. For example, the cover may include an inwardly protruding feature that forces the wick toward the liquid in the liquid chamber while the cover is in place. When the cover is removed, the wick can tend to revert back to its relaxed state (e.g., the wick can return to an unforced flat position or state), and allow air to diffuse through it into the liquid chamber, desirably pulling at least some liquid that may be present on the exposed surface of the wick back into the fluid chamber to, for example, reduce or prevent initial leaks or drips when the air freshener is first activated. Such a configuration works, for example, because the wick moving from the deformed position to the relaxed position will tend to reduce the pressure within the liquid chamber with respect to the external environment, and thus air will be pulled through the wick toward the liquid chamber in order to equalize the pressure between the liquid chamber and the external environment.

Some embodiments of air fresheners disclosed herein are also configured to reduce or limit the effect of the external environment on the liquid reservoir or chamber that holds the supply of volatile liquid. For example, various embodiments disclosed herein include a separate reservoir body (sometimes referred to as an insert and/or an airless insert) that is desirably positioned within a separate air chamber or cavity, with the external surface of the liquid reservoir body not being directly exposed to the external environment (e.g., any exposure to pressure changes in the external environment on the exterior surface of the reservoir body is through pressure changes within the surrounding air chamber that is in fluid communication with or vented to the external atmosphere). In such a configuration, the liquid chamber of the reservoir body or insert may desirably be completely full (or substantially completely full, such as 90%, 95%, or more full) with volatile liquid before the cover is removed to activate the air freshener. Accordingly, little to no air may be present within the liquid chamber of the reservoir body before the cover is removed that allows air to diffuse through the wick into the liquid chamber of the reservoir body. Having little or no air present within the liquid chamber—at least prior to removal of the cover to activate the air freshener—can help to reduce negative effects of external environment barometric pressure changes. One reason for this is that, the higher the percentage of the liquid chamber volume that is air (as opposed to liquid), the more external environment pressure changes will cause variances in the evaporation rate of the liquid through the wick. Further, having the reservoir body positioned within a separate vented air chamber defined by an outer vessel of the container can be beneficial, such as because changes in the barometric pressure of the external environment may then desirably have little to no effect on the external vessel wall, and/or deformation of the external vessel wall caused by handling (e.g. by maintenance personnel or other users) will have little to no effect on the reservoir body wall and/or the pressure within the liquid chamber.

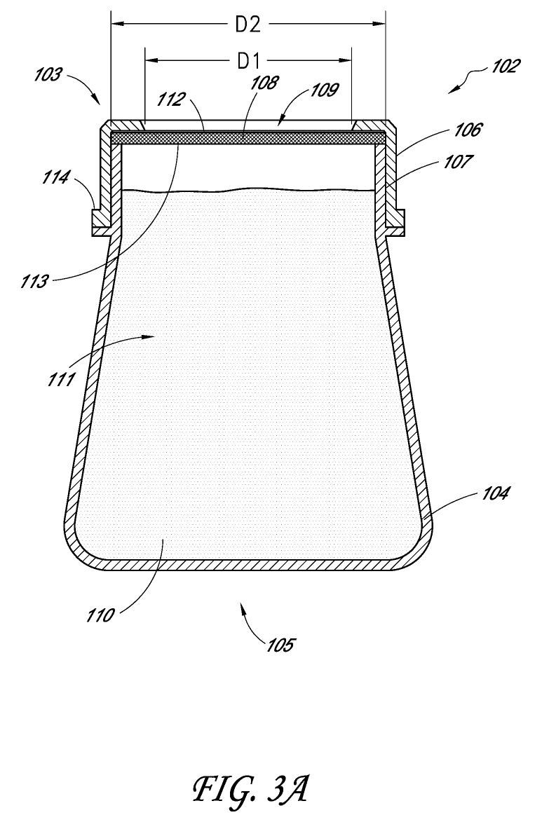

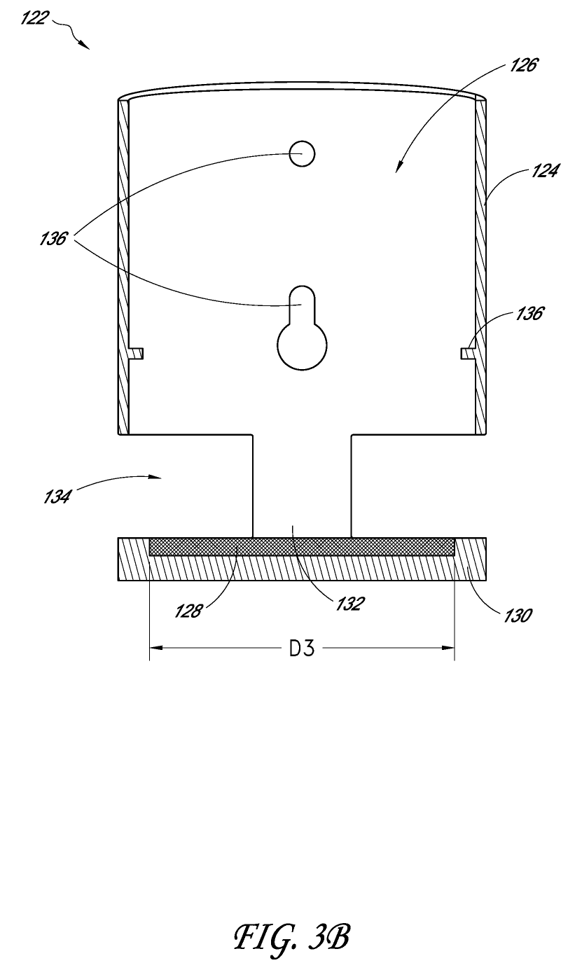

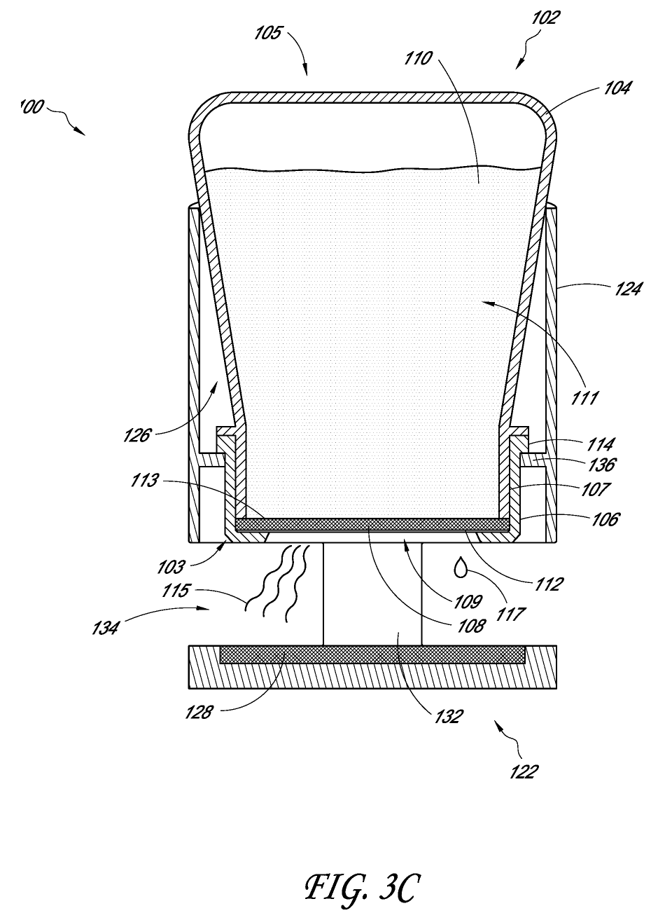

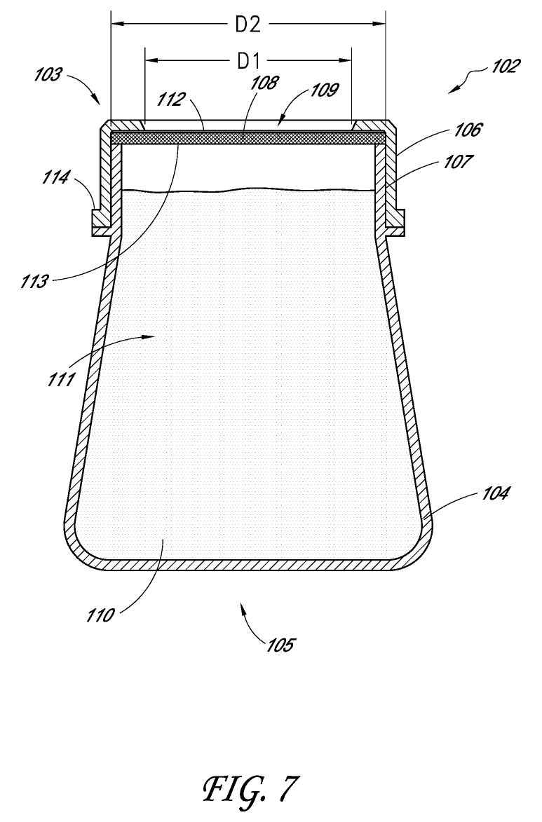

Additionally, in some embodiments, a barrier may be included between the wick and the volatile liquid, to prevent or limit the volatile liquid from flowing into the wick until a user removes the barrier, punctures the barrier, or otherwise causes the barrier to no longer prevent or limit the volatile liquid from flowing into the wick. For example, FIG. 7 is a schematic cross-section view of a fragrance container 702 that is similar to the fragrance container 102 of FIG. 3A (described below), with the same or similar reference numbers referring to the same or similar features. A difference in FIG. 7 is that a barrier 716 is positioned between the wick 108 and the volatile liquid 110. The barrier 716 may comprise a sheet, disc, seal, or the like comprising a material that is non-permeable to the volatile liquid, such as a plastic, polymer, metal foil, and/or the like. The barrier 716 may be removed, torn, punctured, or the like in order to activate the air freshener (e.g., in order to allow volatile liquid to flow into the wick). In some embodiments, the volatile liquid comprises a formulation or blend of a plurality of fragrant chemicals that each have a vapor pressure within a relatively narrow range. Such a formulation can lead to even more consistent fragrance release over time.

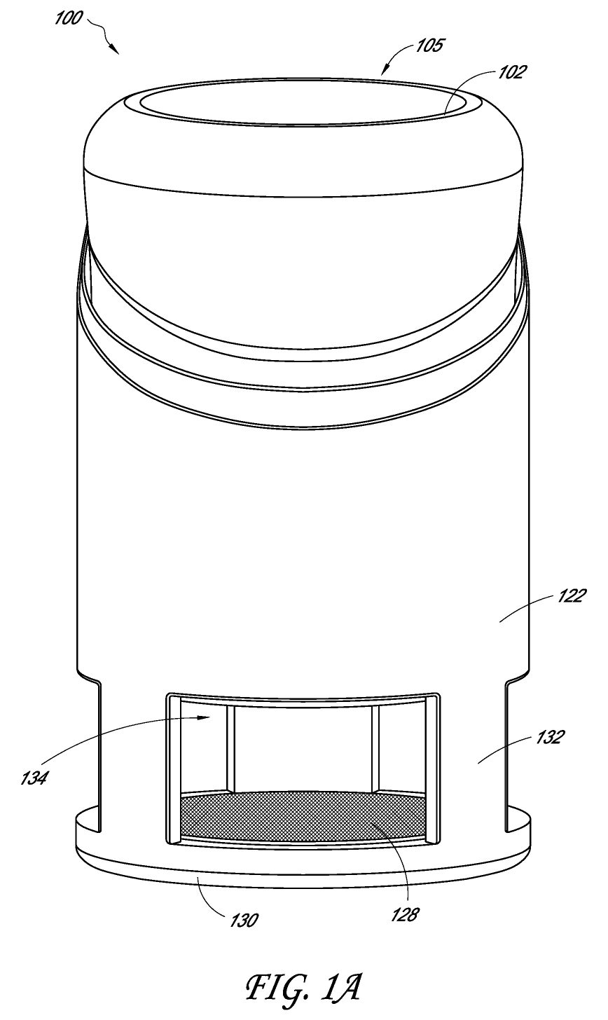

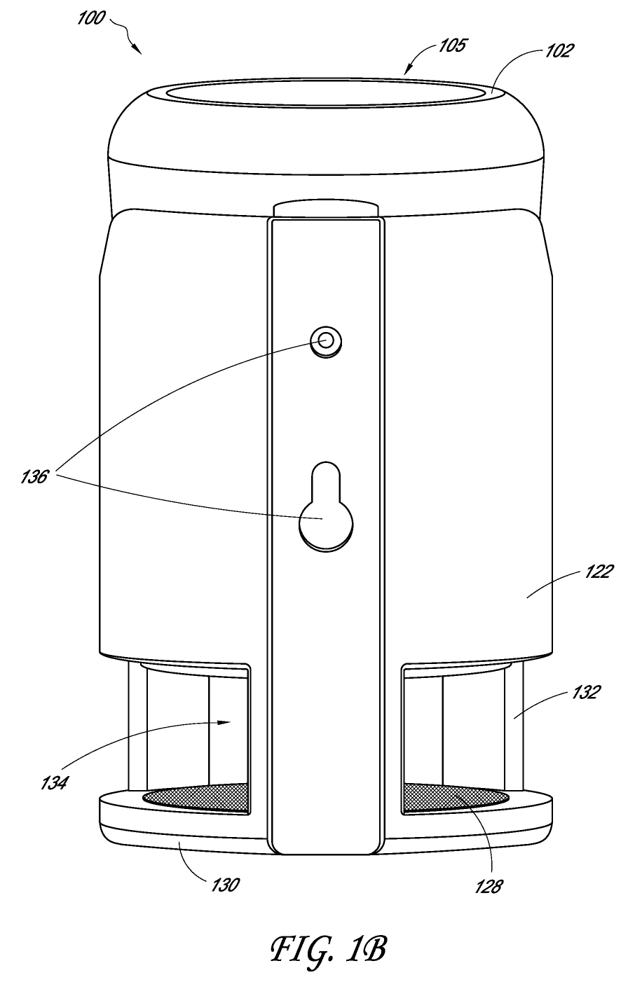

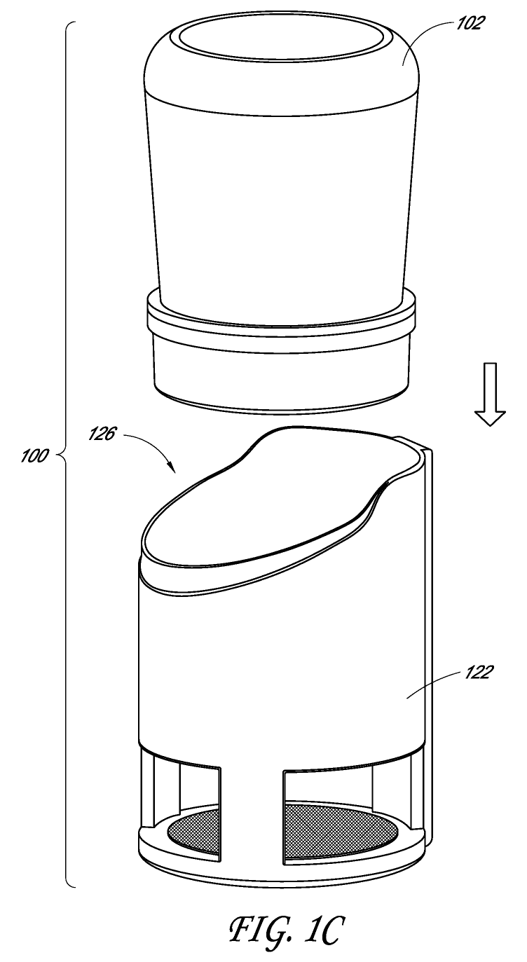

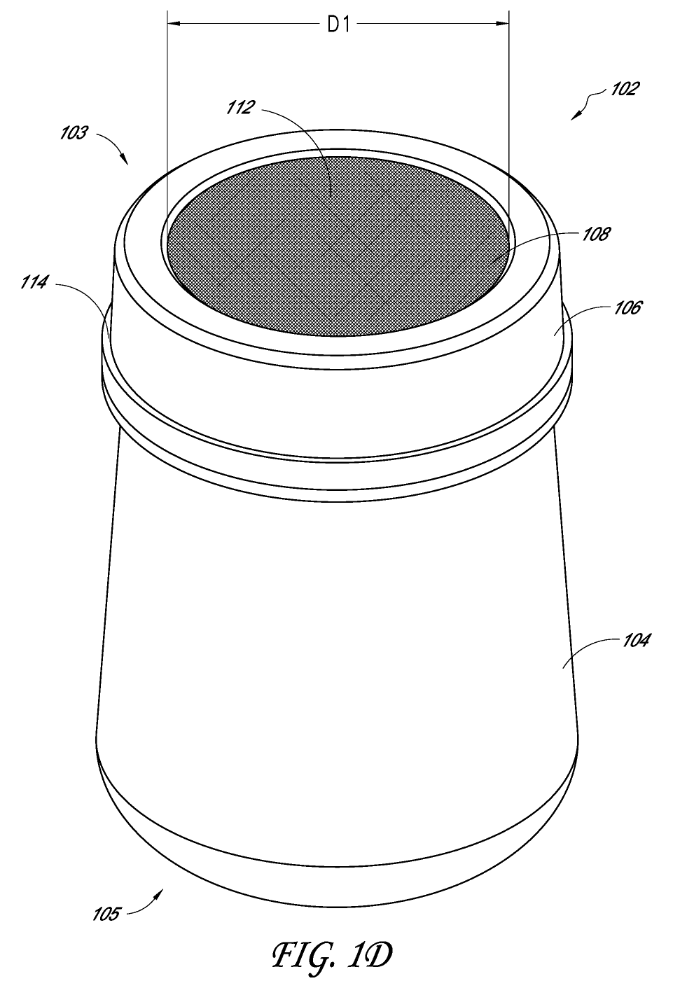

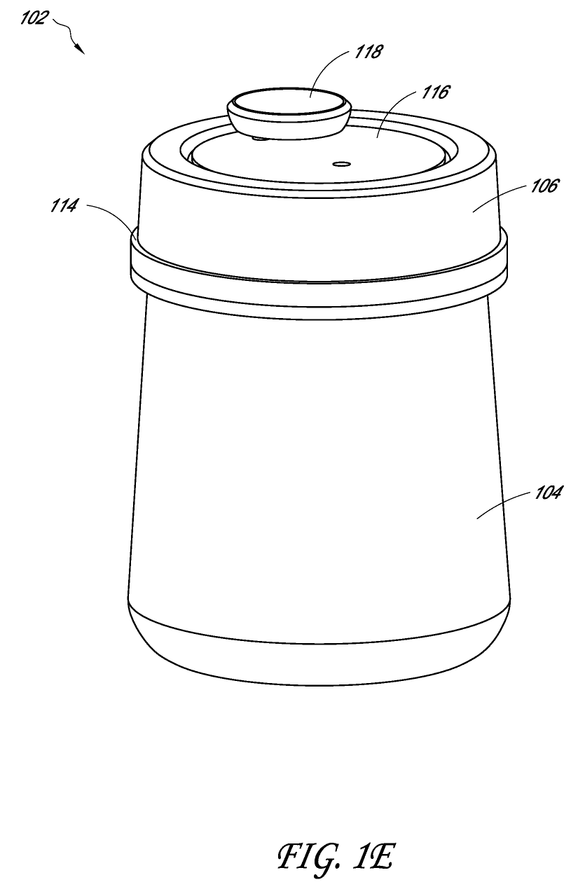

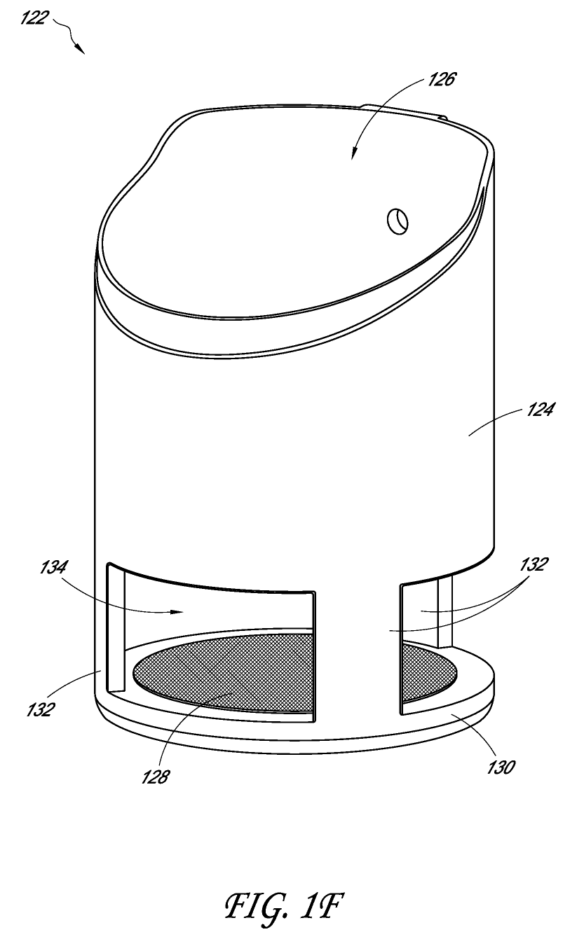

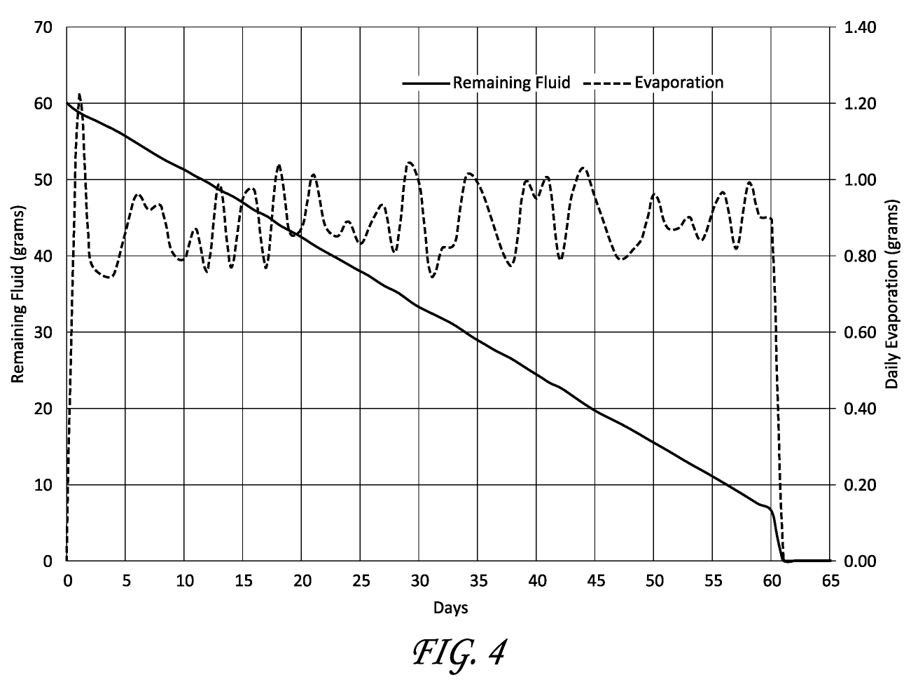

FIG. 4 is a chart showing an example amount of evaporation over time for an exemplary embodiment of an inverted and/or gravity fed air freshener as disclosed herein (such as, for example, the air freshener 100 of FIG. 1A, which is described in greater detail below). In this embodiment, the air freshener begins with 60 grams of a volatile liquid blend positioned therein and is designed to last for approximately 60 days, with the amount of volatile liquid being lost through evaporation through the wick being approximately 0.9 grams per day. With reference to FIG. 4, the x-axis shows the number of days, from 0 to 65, since unsealing and inverting the air freshener fragrance container. The solid line shows the remaining amount of volatile liquid in the fragrance container, with reference to the first y-axis shown on the left side of the chart. The dashed line shows the amount of evaporation through the wick each day, with reference to the second y-axis shown on the right side of the chart.

As can be seen in this chart, on day one, the air freshener experiences a somewhat higher amount of evaporation (approximately 1.2 grams) as the wick loads up or becomes saturated with the volatile liquid blend. After the wick is saturated or loaded up with the volatile liquid, and the fragrance container remains inverted to keep the volatile liquid in contact with the wick, the daily amount of evaporation remains relatively constant, in this case being between approximately 0.75 g and 1.05 g per day. At least some of the daily variation may be due to, for example, changes in barometric pressure in the environment, which may cause more or less evaporation through the wick as the pressure within the fragrance container equalizes with the external barometric pressure of the environment. Finally, at about day 60, a steep drop off in the daily evaporation can be seen as the fragrance container runs out of volatile liquid.

One way various embodiments disclosed herein are able to maintain a relatively consistent output of fragrance over time is due to the volatile liquid being forced against the wick relatively consistently over time by the force of gravity. This can lead to a more consistent fragrance output than, for example, an air freshener that utilizes a heating element that will have variations in the amount of heat put into the liquid, or an air freshener that utilizes an atomizer that, for example, sprays fragrance into an environment on preset intervals or the like.

Another way various embodiments disclosed herein are able to maintain a relatively consistent output of fragrance over time is due to some embodiments having an airless insert or separate reservoir body that desirably is initially completely loaded with a volatile liquid, with no or very little air present in the liquid chamber. Although over time as the volatile liquid evaporates through the wick air will be introduced into the liquid chamber to equalize pressures, the percentage of the volume of the liquid chamber that is air versus liquid will remain lower for longer than if the liquid chamber initially had some air in it. By keeping the percentage of volume of the liquid chamber that is air lower for longer, this can reduce the effects of external barometric pressure changes. For example, some embodiments have a liquid chamber (e.g., within a reservoir body or insert) that has a volume, with at least 90%, 95%, 96%, 97%, 98%, 99% or 100% of the volume being the volatile liquid (while the cover is installed—before the air freshener is activated). In some embodiments, during manufacturing, the liquid chamber may have at least some air within it, but all or most of that air is forced out through the wick when a cover is installed that forces or deforms the wick inward (as described below with respect to, for example, air freshener container 802)—reducing the volume of the liquid chamber and expelling the air.

Another way various embodiments disclosed herein are able to maintain a relatively consistent output of fragrance over time is that various embodiments utilize a volatile liquid that is a chemical formulation or blend comprising a plurality of chemicals that each have a similar vapor pressure (e.g., that each have a vapor pressure within a relatively small range of each other). In some embodiments, the vapor pressure difference between the most volatile chemical and the least volatile chemical in the blend may be less than 3mmHg, less than 2mmHg, less than 1.5mmHg, less than 1mmHg, less than 0.5mmHg, or less than 0.4mmHg. In some embodiments, the vapor pressure difference between the most volatile chemical and the least volatile chemical in the blend may be between 3mmHg and 0.4mmHg, between 2.5mmHg and 0.4mmHg, between 2mmHg and 0.4mmHg, between 1.5mmHg and 0.4mmHg, between 1mmHg and 0.4mmHg. In some embodiments, the vapor pressure difference between the most volatile chemical and the least volatile chemical in the blend may be between about 3mmHg and about 0.4mmHg, between about 2.5mmHg and about 0.4mmHg, between about 2mmHg and about 0.4mmHg, between about 1.5mmHg and about 0.4mmHg, between about 1mmHg and about 0.4mmHg. For example, some embodiments may utilize a volatile liquid formulation that is comprised of a plurality of volatile chemicals all having a vapor pressure within 0.4mmHg (or about 0.4mmHg) from one another, within 1mmHg (or about 1mmHg) from one another, within 2mmHg (or about 2mmHg) from one another, within 3mmHg (or about 3mmHg) from one another, or the like. This can be beneficial, for example, because volatile chemicals that have significantly different vapor pressures are generally comprised of molecules of significantly different sizes, and may all have different levels of resistance to flowing through a particular wick material.

For example, fragrance formulations or blends typically comprise a plurality of chemicals that each generate a different aroma, and that may each be classified as a top note, middle note, or base note. Chemicals corresponding to each of these types of “notes” tend to have significantly different vapor pressures, and significantly different molecule sizes. If a volatile liquid formulation or blend is utilized that includes one or more chemicals having a relatively low vapor pressure (corresponding to relatively large molecules) and one or more chemicals having a relatively high vapor pressure (corresponding to relatively small molecules), the chemical having the relatively lower vapor pressure may tend to clog the pores of the wick material and, over time, gradually inhibit or lower the ability of the higher vapor pressure chemical to pass through the wick. In such a situation, over time, the amount of volatile liquid being evaporated through the wick will decrease day-to-day, resulting in a less effective air freshener as time goes on (e.g., due to less fragrance being released as time goes on). Further, over time, the aromatic characteristics of the chemicals being evaporated may change, causing the smell experienced by a user of the air freshener to change. By utilizing a chemical formulation or blend that comprises a plurality of volatile chemicals each within a relatively narrow range of vapor pressures, (1) the total amount of volatile liquid being evaporated through the wick will be more stable over time (i.e. the potency of the aroma will be more stable), and (2) the aromatic characteristics of the evaporated chemicals will be more stable over time (i.e. the perceived “smell” of the released fragrance will tend not to change significantly over time). In some embodiments, the volatile liquid is a chemical formulation or blend comprising at least two, three, four, or more chemicals each having a vapor pressure within a relatively narrow range, including within any of the vapor pressure ranges disclosed herein.

As mentioned above, aromatic chemicals can produce fragrances or aromas that include a plurality of “notes” classified as top notes, middle notes, or base notes. Top notes are typically fresh smelling fragrances, such as lemongrass, middle notes are characteristic of heady smelling flowers, like irises or orchids, and base notes are typically animalistic, such as musk. Top notes are sometimes referred to as head notes and will typically be the first aroma a person experiences when smelling a particular fragrance. Top notes evaporate more quickly than other notes, and some examples of top notes include citrus scents, such as lemon, orange, and bergamot, and lighter smelling flowers, such as rose and lavender. Anise and basil may also be considered top notes. Middle notes may also be referred to as heart notes. Middle notes or heart notes may be intended to, for example, reinforce the top notes and/or act as a buffer for base notes, which may be less pleasant if they are provided on their own. Examples of middle notes include aromatic floral oils, such as geranium, jasmine, neroli, and ylang-ylang, and also black pepper, cardamom, pine, and cinnamon. Base notes may tend to form a foundation of a fragrance. Base notes are rich and longer-lasting, with examples including moss, patchouli, musk, amber, vanilla, and woody notes such as sandalwood or cedarwood. In some embodiments of air fresheners disclosed herein, the fragrance formulation or blend includes chemicals providing at least two, three, or more distinct aromas from within a particular classification (e.g., two, three, or more top notes, two, three, or more middle notes, or two, three, or more base notes). In some embodiments, of air fresheners disclosed herein, the fragrance formulation or blend includes chemicals providing at least two, three, or more distinct aromas, with at least some of the aromas coming from different classifications (e.g., one or more top notes with one or more middle notes, one or more middle notes with one or more base notes, or one or more of each of a top note, middle note, and base note). Desirably, although the formulation or blend may include chemicals providing aromas from different classifications, the chemicals may be specifically chosen to have relatively similar vapor pressures (e.g., vapor pressure differences within any of the ranges disclosed herein).

In addition to the benefits described above associated with keeping the vapor pressures of the various chemicals of a volatile liquid formulation to within a relatively narrow range, such a chemical formulation can also allow the type and/or size of wick to be tuned to a particular chemical formulation, in order to tune the air freshener to provide a particular level of fragrance output over a particular period of time. For example, if the average volatility of a second chemical formulation is higher than that of a first chemical formulation, the air freshener using the second chemical formulation may be designed to use a wick that comprises a lower exposure area (e.g., the amount of surface area exposed to the environment), and/or to use a wick material that has a higher resistance to evaporation therethrough.

### Example Air Freshener

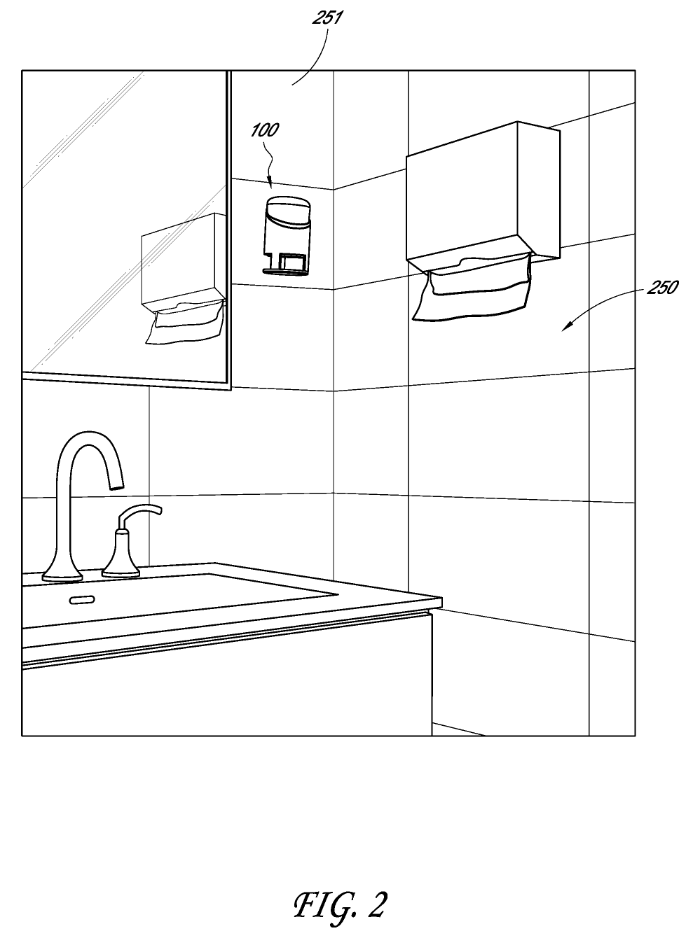

FIGS. 1A-1F illustrate an example embodiment of an air freshener 100 that can include some or all of the benefits described above. The air freshener 100 includes a fragrance container 102 that can be held in an inverted orientation by a holder 122. FIGS. 1A, 1B, and 3C depict the container 102 held in the inverted orientation by the holder 122, with FIG. 1A being a front perspective view, FIG. 1B being a back perspective view, and FIG. 3C being a cross-section view. The container 102 may be removably coupleable to the holder 122, and FIG. 1C is an exploded view showing the container 102 in the inverted orientation being inserted into a cavity 126 of the holder 122 (e.g., a cavity defined by a generally annularly shaped wall of the holder 122). FIG. 1D is a perspective view of the container 102 in an upright or non-inverted orientation, and FIG. 1E shows the container 102 with a cover 116 attached thereto. FIG. 1F is a perspective view of the holder 122 by itself, and FIG. 2 shows the air freshener 100 in an example environment (in this case, being attached to a wall 251 of a bathroom environment 250). Additionally, FIGS. 3A and 3B are schematic cross-sectional views of the fragrance container 102 and the holder 122, respectively.

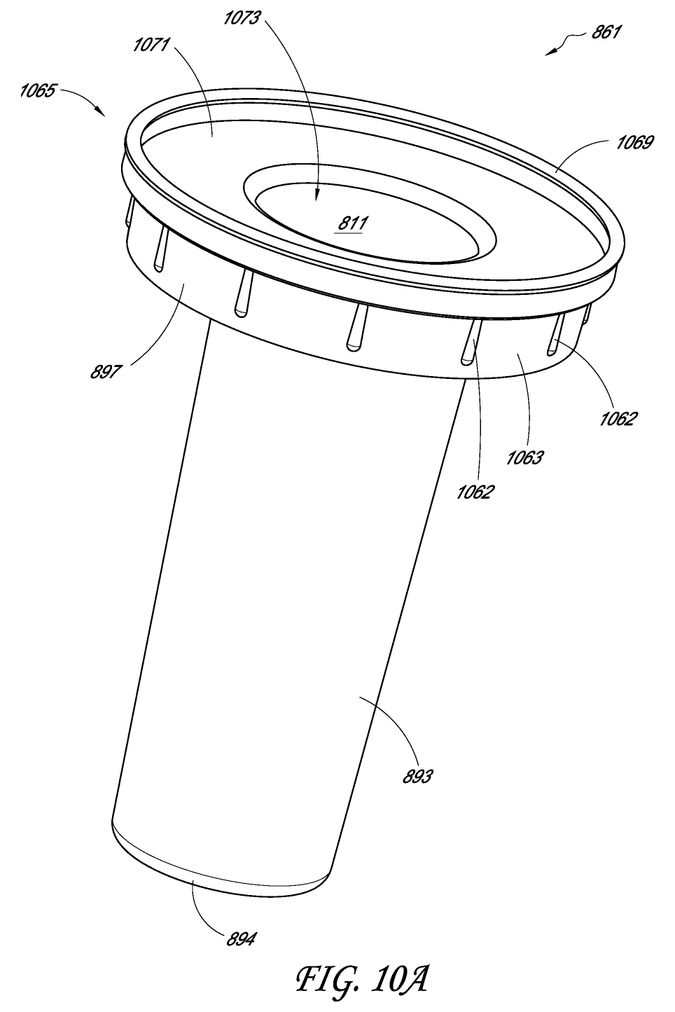

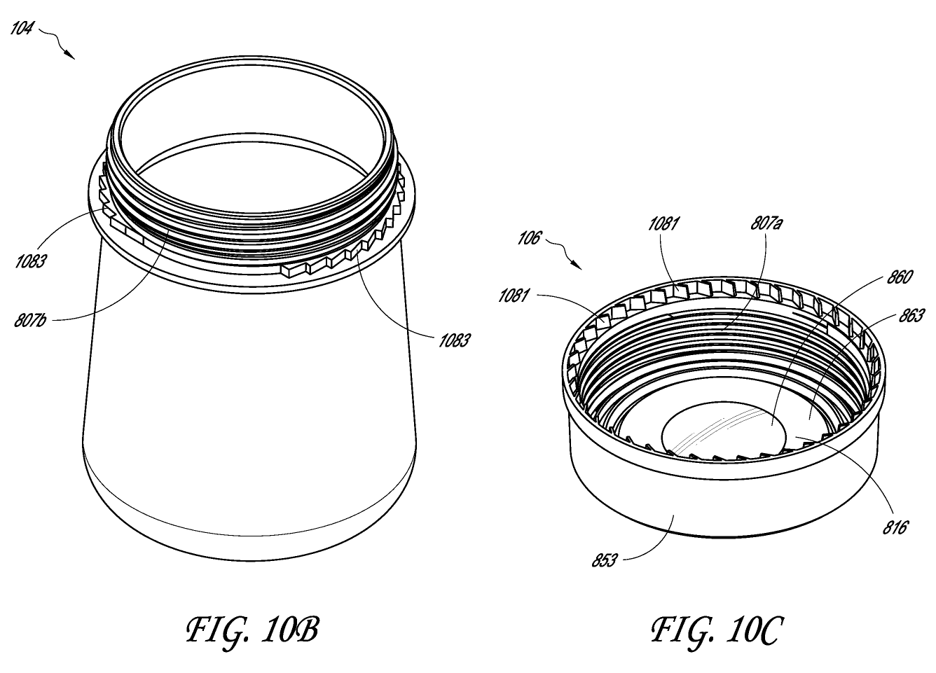

With reference to FIGS. 1D, 3A, and 3C, the container 102 comprises a vessel 104 that defines a cavity 111 containing a volatile liquid 110 therein. In some embodiments, as described above, the volatile liquid 110 is a blend of fragrance chemicals each having a vapor pressure within a relatively narrow range. At the top end 103 of the container 102 is a lid 106. The lid 106 in this embodiment is coupled to the vessel 104 using a thread 107, but other embodiments may couple the lid 106 to the vessel 104 differently, such as by using a snap fit, an adhesive, plastic welding, and/or the like. In some embodiments, it may be desirable to attach the lid 106 to the vessel 104 in a relatively nonremovable fashion, such as to avoid an end user accidentally removing the lid and spilling the volatile liquid 110. For example, the vessel 104 and lid 106 may implement the locking mechanism shown in FIGS. 10B and 10C, which include interlocking teeth 1081 and 1083. In some embodiments, however, it may be desirable to allow an end user to remove the lid, such as to remove a barrier between the wick 108 and the volatile liquid 110 to facilitate activating the air freshener, as described above with reference to FIG. 7.

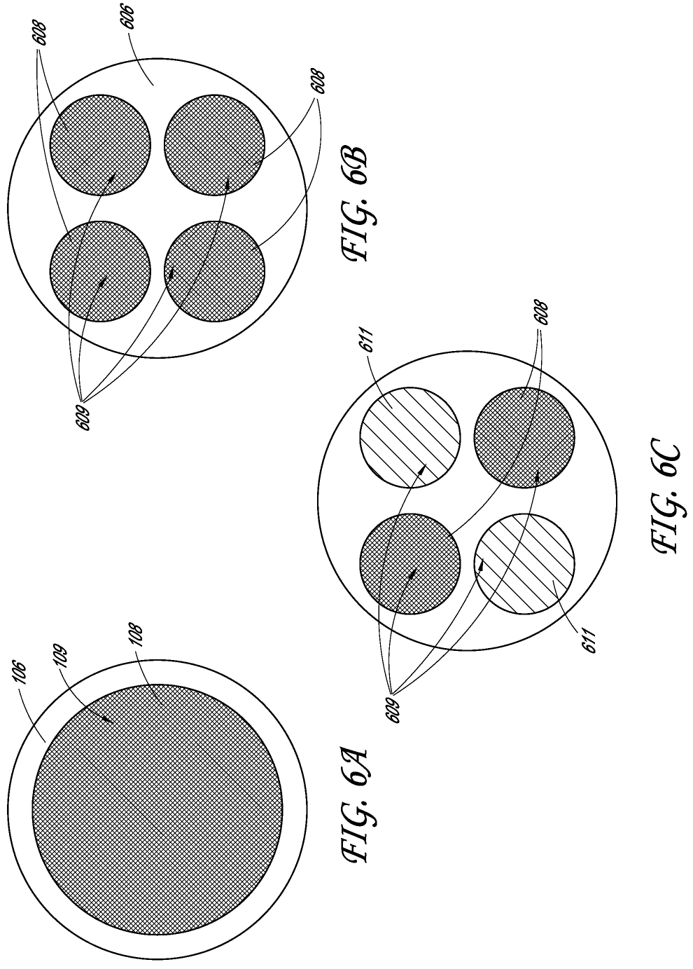

The container 102 further comprises a wick 108. In this embodiment, the wick 108 comprises a sheet of wick material, such as a pulp-based wicking material (e.g., a wood pulp wick or a paper pulp wick), that is formed in a circular or disc shape. Other embodiments may use different material for the wick (such as, for example, engineered plastics), may shape the wick differently, may utilize more than one wick (see, for example, FIGS. 6B and 6C described below), and/or the like. Notably, the wick 108 of the container 102 is of a sheet form (e.g., a form having a relatively high width-to-thickness ratio, such as, for example, at least 5 to 1, at least 10 to 1, at least 20 to 1, at least 30 to 1, at least 40 to 1, at least 50 to 1, 75 to 1, 100 to 1, between 5 to 1 and 100 to 1, between 10 to 1 and 75 to 1, or the like) as opposed to a tubular or elongate cylinder form, and the sheet is desirably oriented such that the primary transfer of liquid 110 from the cavity 111 to the ambient environment occurs directly across the thickness of the wick (e.g., from interior or wetted surface 113 to exterior, evaporative, or exposed surface 112). In other words, the wick 108 is wider than it is thick (the thickness being the distance between the interior surface 113 and the exterior surface 112), with the interior surface 113 in direct contact with the liquid 110 in the cavity 111 when inverted and the exterior surface 112 in contact with environmental air. Further, the wick 108 is desirably positioned such that the entire inner side of the wick (e.g., interior or wetted surface 113), or at least a majority of the inner side of the wick (such all of the inner side of the wick except for areas of the wick that are covered by the wall of the vessel 104) will remain directly in contact with the liquid 110 as long as the container 102 remains in the inverted orientation (e.g., the orientation shown in FIGS. 1A and 3C). In some embodiments, at least 75%, 80%, 85%, 90%, or 95% of the inner side of the wick is positioned to remain directly in contact with the liquid 110 as long as the container 102 remains in the inverted orientation.

The wick 108 desirably comprises a pulp-based wicking material (i.e. a material comprising cellulose fibers) having a density of 4.8 lbs./mil and a thickness of 85 mils. Such a material has been found through testing to have desirable wicking characteristics, such as to allow an air freshener using a chemical blend comprising a plurality of chemicals with a vapor pressure difference between a most volatile chemical of the plurality of chemicals and a least volatile chemical of the plurality of chemicals of no greater than 1 mmHg to last a relatively long time before the liquid is completely evaporated. An example of one such material is sold under the product name AF-085 Blotter by Robert Wilson Paper Corporation of Lynbrook, New York, for use as a absorbent media. In some embodiments, the wick 108 comprises a pulp-based wicking material having a density within a range of 4.6-5.0, 4.4-5.2, 4.2-5.4, or 4.0-6.0 lbs./mil. In some embodiments, the wick 108 comprises a pulp-based wicking material having a thickness within a range of 80-90, 70-100, or 60-140 mils. In some embodiments, the wick 108 comprises a pulp-based wicking material having a basis weight within a range of 390-435 lbs./3000 ft², such as 415 lbs./3000 ft². In some embodiments, the wick 108 comprises a pulp-based wicking material having a basis weight within a range of 370-460, 350-500, or 300-550 lbs./3000 ft². In some embodiments, the wick 108 comprises a pulp-based wicking material having a holding capacity of 365%. In some embodiments, the wick 108 comprises a pulp-based wicking material having a holding capacity of at least 300%. In some embodiments, the wick 108 comprises a pulp-based wicking material having a holding capacity within a range of 200%-500%, 250%-450%, or 300%-400%. In some embodiments, the wick 108 comprises a pulp-based wicking material having a moisture content (prior to coming into contact with the liquid 110) of 3.8%. In some embodiments, the wick 108 comprises a pulp-based wicking material having a moisture content (prior to coming into contact with the liquid 110) within a range of 2.5-4.5%, 2.0-5.0%, or 3.0-6.0%. In some embodiments, the wick 108 comprises a pulp-based wicking material having a tensile strength (MD) of 60 lbs./linear inch. In some embodiments, the wick 108 comprises a pulp-based wicking material having a tensile strength (MD) within a range of 40-80, 30-90, or 20-100 lbs./linear inch. It should also be noted that, although engineered plastics could potentially be used as a wick, testing of air fresheners similar to those disclosed herein has shown pulp-based wicking material to be more effective in an inverted gravity-fed air freshener.

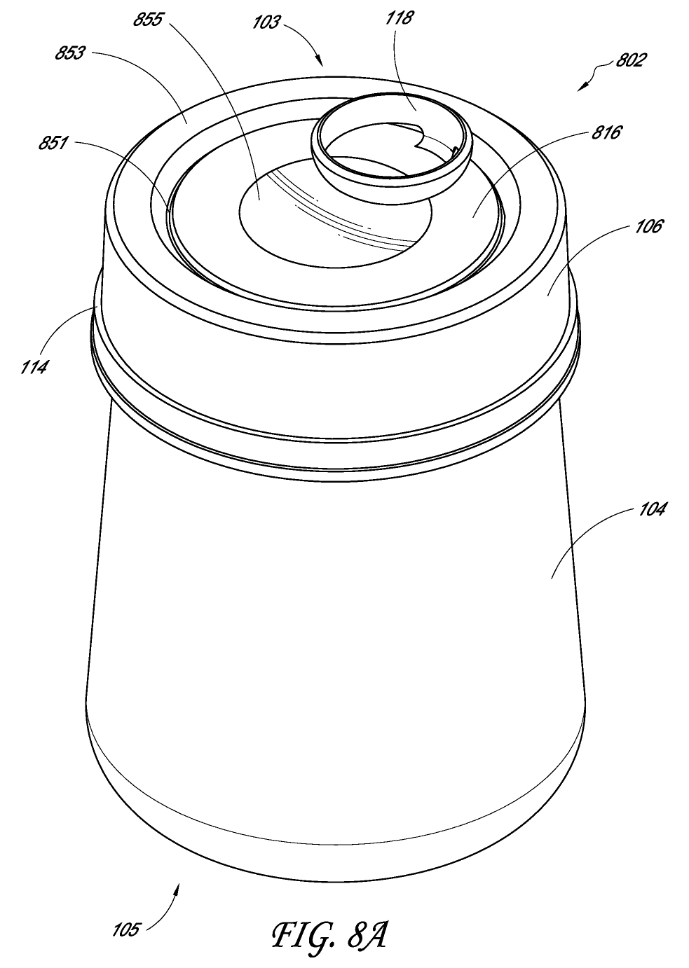

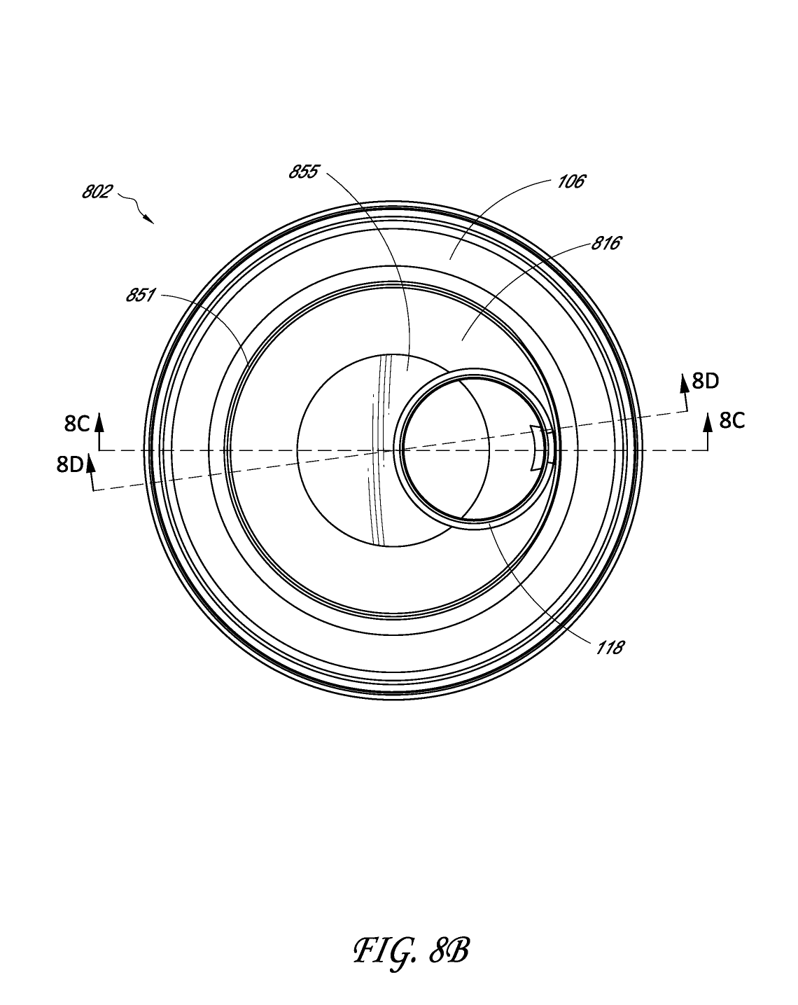

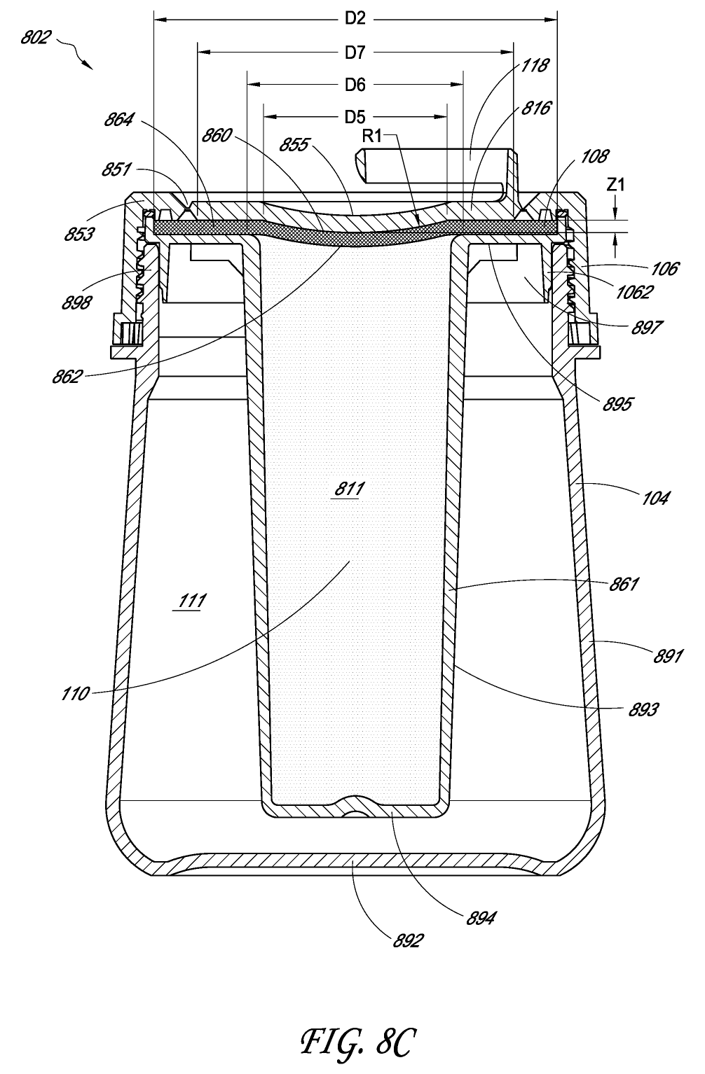

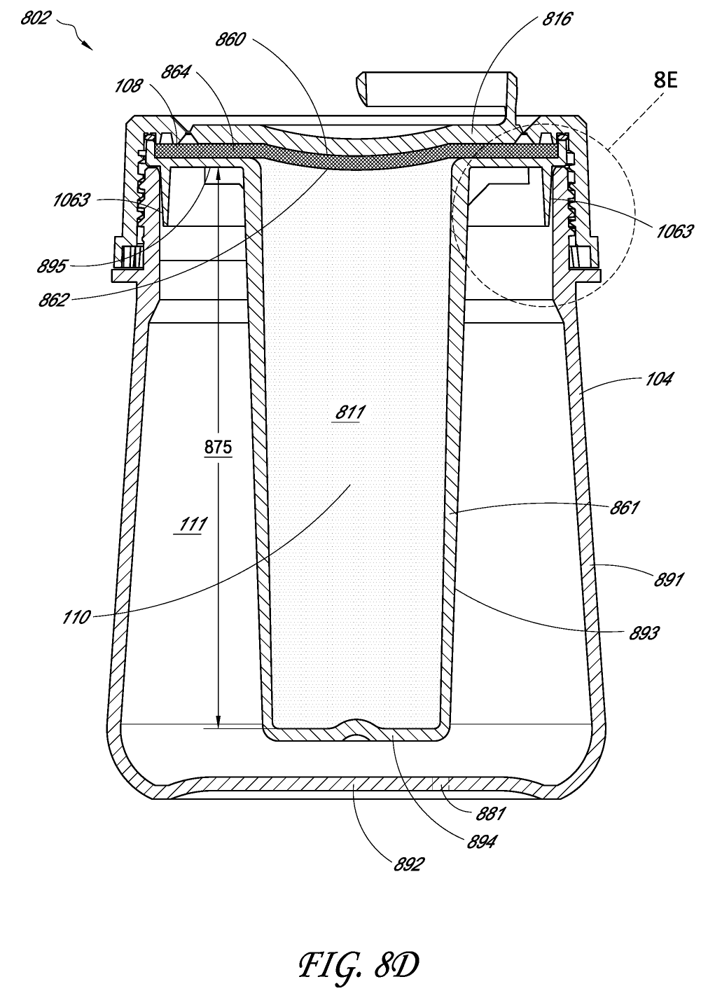

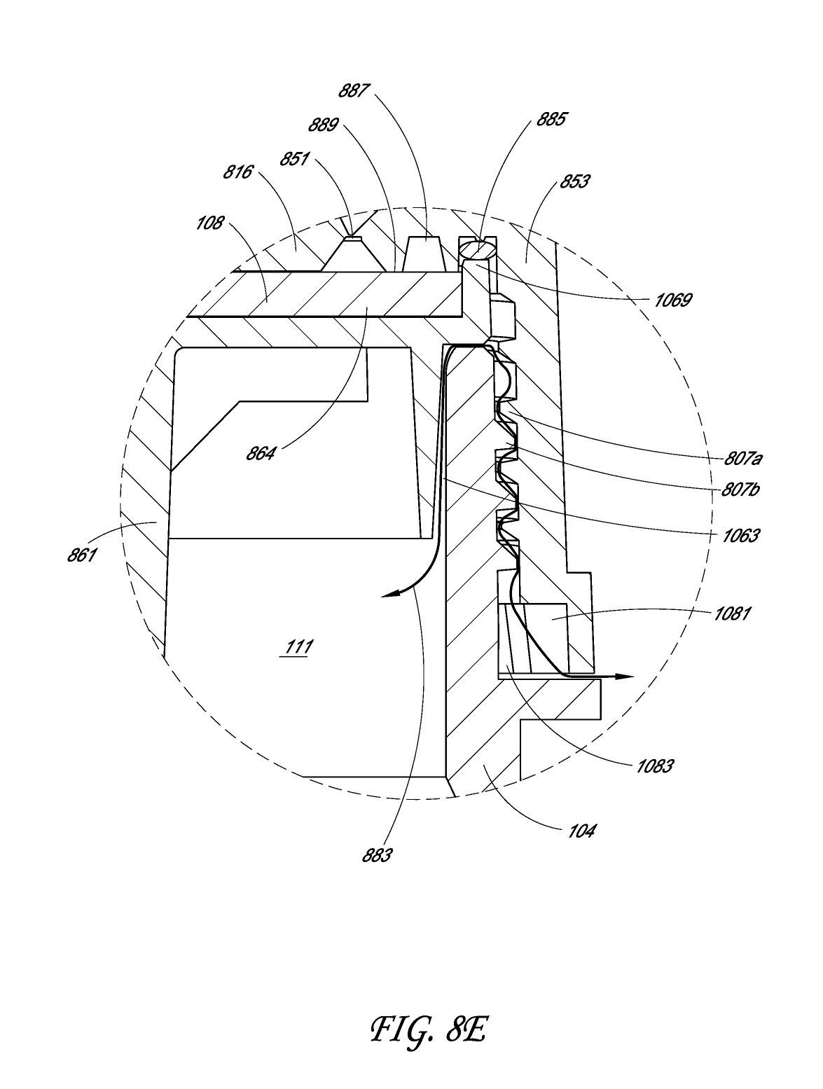

With continued reference to FIGS. 3A and 3C, in this embodiment, the wick 108 is desirably captured between and/or held in place between the lid 106 and vessel 104. In some embodiments, the lid 106 may include one or more features that help to retain an outer edge, rim, or portion of the wick 108 in place, such as the protrusion 889 and/or cavity 887 shown in FIG. 8E and described below. Further, the lid 106 comprises an opening 109 that exposes a portion of the wick 108 to the external environment (e.g., exterior, evaporative, or exposed surface 112). Desirably, the container 102 is configured such that the liquid 110 disposed therein is sealed off from the external environment other than through the exposed surface 112 of the wick 108. In other words, the pressure within cavity 111 is self-regulating through the wick 108. For example, when the pressure within cavity 111 changes with respect to the exterior ambient environmental pressure (e.g., due to temperature changes, changes in the volume of liquid left in the cavity 111, other changes in pressure within the cavity 111, changes in the ambient environmental pressure, and/or the like), desirably the only fluid flow path to tend to equalize the pressures is through the wick 108. In other words, there is desirably no additional pressure equalization flow path (such as an air vent), other than through the wick. This can be desirable, for example, to help extend the life of the air freshener, such as by eliminating a flow path through which the liquid 110 may otherwise undesirably be lost (such as through leakage and/or evaporation). It should be noted that, in some embodiments, the container 102 may include a pressure equalization flow path other than through the wick. Such an embodiment is generally less desirable than an embodiment without such a flow path, however, for at least the reasons provided above. In alternative embodiments that include multiple wicks in contact with the liquid 110 (see, for example, FIGS. 6B and 6C), the liquid 110 is desirably sealed off from the external environment other than through the exposed surfaces of the multiple wicks. Similarly, the total exposed surface area of the multiple wicks is desirably within the ranges described herein with reference to a single wick.

With continued reference to FIG. 3A, in this embodiment, the wick 108 comprises an outer diameter D2, but only the exposure area or surface 112 defined by diameter D1 of the opening 109 in the lid 106 is exposed to the environment. As a non-limiting example, the outer diameter D2 may be approximately 65mm, while the exposed diameter D1 may be approximately 50mm. The size of the exposure or exposed area 112 can at least partially control the rate of fragrance release (e.g., the rate of evaporation of volatile liquid 110 through the wick 108) when the container 102 is put in an inverted position and gravity forces the liquid 110 against the wick 108. FIG. 3C shows the container 102 in the inverted position, with wavy lines 115 representative of the evaporation from exposed area 112 into the ambient environment.

FIGS. 1D, 3A, and 3C also show that the container 102 includes a ledge 114 (in this case as part of the lid 106, but the ledge 114 may alternatively be positioned and/or shaped differently). This ledge 114 is desirably shaped such that, when the bottom end 105 of the container 102 is positioned upward (e.g., the container 102 is inverted), and the container 102 is inserted into the cavity 126 of the holder 122 (see FIG. 1C), a mating ledge 136 within the holder 122 (see FIG. 3B) can support the inverted container 102 (as shown in FIG. 3C). Desirably, the container 102 is positioned with the wick 108 (e.g., a first or primary wick) spaced apart above a second or secondary wick 128, as shown in FIG. 3C, and as further described below.

With reference to FIG. 3B, this figure shows a cross-sectional view of the holder 122. The holder 122 comprises a main body 124 that defines a cavity 126 for positioning therein of at least a portion of the inverted container 102 (as shown in FIGS. 1A-1C). For example, the main body 124 comprises a generally annularly shaped wall that at least partially defines the cavity 126. The main body 124 further comprises a plurality of fastener apertures 136 that can be used to, for example, mount the holder 122 to a wall, as shown in FIG. 2.

FIGS. 1A, 1B, 3B, and 3C also show that the holder 122 comprises a second body or drip collection member 130 that is supported below the main body 124 by a support structure comprising a plurality of support legs or support members 132 extending therebetween. In this embodiment, the second body or drip collection member 130 is generally circular or disc-shaped, with three support legs or members 132 spaced apart from one another and connecting the second body or drip collection member 130 to the main body 124. Other embodiments may shape the second body or drip collection member differently and/or use more or fewer and/or differently shaped or positioned support legs or members 132. In some embodiments, the legs or support members 132 and the ledge 136 may individually or collectively be referred to as support structures.

The holder 122 further comprises a second wick 128 supported by the drip collection member 130 (e.g., in this embodiment, positioned within a cavity of the drip collection member 130). When the inverted container 102 is held in place by the holder 122 (e.g., with ledge 114 of the container 102 engaged with ledge 136 of the holder 122, as shown in FIG. 3C), the container 102 is desirably held above the drip collection member 130 and the second wick 128, such that a space 134 is formed therebetween. The space 134 can allow for evaporation of the volatile liquid 110 through the first wick 108 of the container 102 into the external environment (as indicated by wavy lines 115 of FIG. 3C).

Although there are many benefits to a gravity fed volatile liquid air freshening system (as discussed above), one challenge of such a system is that the barometric pressure of the external environment will tend to vary over time. As discussed above, one way to address such variations is to add a separate flow path (e.g., a flow path other than through the wick 108) through which pressure can equalize. Such a solution can cause other problems or undesirable effects, however, such as reduction in useful life of the air freshener due to loss of liquid through such a flow path. Accordingly, various embodiments disclosed herein, including the embodiment shown in FIG. 3C, desirably do not include such a pressure equalization flow path. This can lead to some fluctuations in the amount of liquid evaporated through the wick 108 of the container 102, however, such as is shown in the example evaporation chart of FIG. 4. Additionally, when the barometric pressure of the external environment drops with respect to the pressure within the vessel 104 of the fragrance container 102, in some cases the requirement to equalize pressure through the wick 108 may cause one or more liquid drops of the volatile liquid 110 to pass through and fall unevaporated from the first wick 108 while the pressure within the vessel 104 is equalizing with the reduced external environment pressure.

In testing of an air freshener similar to the air freshener 100, it has been shown that such environmental changes in pressure can lead to a relatively small amount of the volatile liquid 110 passing through the first or primary wick 108 in a liquid form, such as 2 to 3 drops of liquid, which may not be desirable. In order to address this problem, the second or secondary wick 128 is desirably spaced apart from—but positioned beneath—the primary wick 108, such that the secondary wick 128 will catch any of such drops of liquid that fall from the primary wick 108. For example, the exposed size of the secondary wick in the width direction (such as the exposed diameter of the secondary wick—see diameter D3 of FIG. 3B) may desirably be at least as large as or larger than the exposed size of the primary wick 108 in the width direction (such as the exposed diameter D1 of the primary wick 108—see FIG. 3A), such that when the container 102 is in an inverted position (e.g., the position shown in FIGS. 1A & 3C), a drop falling from any portion of the exposed area 112 of the first or primary wick 108 will tend to be captured by the second or secondary wick 128. FIG. 3C shows an example of such a drop 117 falling from the exposed area 112 of the primary wick 108.

Some embodiments of air fresheners disclosed herein may include the second body or drip collection member 130 (see FIG. 3B) without a second wick 128 positioned therein or coupled thereto. For example, the drip collection member 130 may be a tray that catches or captures any drops of liquid released from the first wick 108 and keeps those drops from impacting the floor or other environmental surfaces beneath the inverted container 102. Alternatively, some embodiments of air fresheners disclosed herein may utilize an absorbent material or mat in place of the second wick 128, in order to absorb any liquid drops that fall from the first wick 108. Using a wick material (e.g., any of the wicking materials discussed above with reference to the first or primary wick 108) for the second or secondary wick 128 can be desirable, however, because a wick material will tend to spread the liquid drop out through capillary action and encourage the liquid drop to relatively quickly evaporate into the environment. Using an absorbent material or just a drip tray, on the other hand, may cause the liquid that drops from the first wick 108 to remain in place for an extended period of time, leading to collection of dirt and debris, potentially leading to spillage, and/or the like. Further, in some embodiments, a user may utilize the same holder 122 at different times with a number of different containers 102 that may each contain a different fragrance chemical formulation. If a secondary wick 128 were not used (e.g., to encourage relatively quick evaporation of any liquid drops), there may be situations when an older fragrance is still present in the drip collection member 130 and contributing at least some aroma to the environment, while fragrance being evaporated from the new container 102 is of a different aroma that may conflict with the aroma being released from the leftover drops of fluid in the drip collection member 130. Accordingly, it can be desirable to utilize a gravity fed volatile liquid air freshening system that utilizes at least two wicks, with the first or primary wick being directly in contact with the volatile liquid and causing the volatile liquid to evaporate therethrough, and with the second or secondary wick being positioned to catch any drops of liquid that fall from the first wick and cause those drops of liquid to be evaporated into the environment.

Returning to FIG. 1E, this figure also illustrates that the container 102 may include a cover 116 that desirably seals the volatile liquid 110 and the wick 108 (see FIG. 3A) off from the environment prior to putting the air freshener in service. For example, the cover 116 may be adhered to the lid 106, the cover 116 may be molded as a part of the lid 106, and/or the like. Further, the cover 116 may include a pull tab or ring 118 that is configured to, for example, enable a user to insert a finger into the ring and pull the cover 116 off of the lid 106. When the cover 116 is pulled off of the lid 106, this will desirably expose the wick 108 to the external environment, and allow the user to activate the air freshener (e.g., by inverting the container 102 and inserting it into the holder 122, as shown in FIG. 1C). In some embodiments, the cover 116 is molded as a part of the lid 106, with a relatively thin seam connecting the cover 116 to a main body of the lid (as shown in, for example, FIG. 8C, which includes a seam 851 between the cover and the main body 853). The seam is desirably thin enough to break apart in response to a user pulling the pull tab 118, thus allowing the cover to be separated from the main body of the lid.

As mentioned above, FIG. 2 shows the air freshener 100 mounted to a wall 251 of a bathroom environment 250. For example, the holder 122 may be affixed to the wall 251 using screws that pass through the apertures 136 (see FIG. 3B). Although wall-mounting may be convenient, the air freshener 100 may also be used without mounting to a wall. For example, the air freshener 100 may be supported on a table, countertop, and/or the like, such as by placing the second body or drip collection member 130 of the holder 122 onto the table, countertop, and/or the like.

### Additionl Air Freshener Example

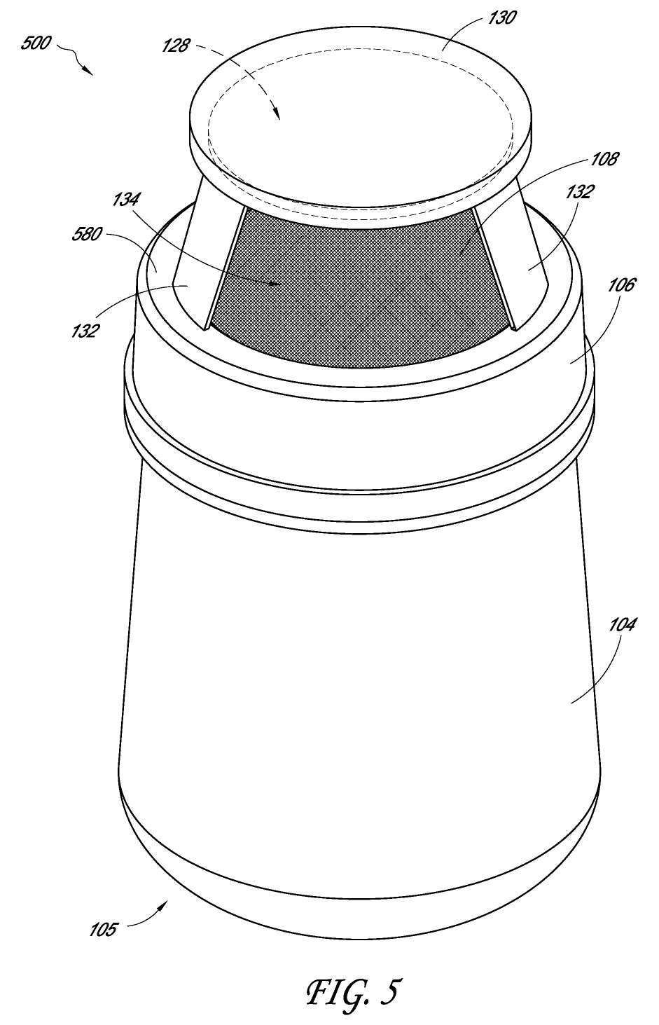

Turning now to FIG. 5, this figure illustrates a schematic example of another embodiment of an inverted gravity fed air freshener 500. This example air freshener is similar to the air freshener 100 described above, and the same or similar reference numbers are used to refer to the same or similar components. The main difference in the air freshener 500 is that the support legs or members 132 and the drip collection member 130 that positions the second wick 128 (similar to second wick 128 of FIG. 3B, shown in FIG. 5 in hidden lines) are integrated into the lid 106 instead of being part of a separate holder (such as holder 122 of FIG. 3B). For example, the support legs or members 132 in this embodiment are part of the lid 106, extending from an upper surface 580 of an annular portion of the lid 106 to the drip collection member 130. In such a configuration, the support legs or members 132 desirably maintain the drip collection member 130 (and thus the second wick 128) in a fixed position with respect to the first wick 108 and vessel 104, regardless of how the air freshener 500 is positioned with respect to the environment (e.g., upright, as shown in FIG. 5, or inverted).

The air freshener 500 may also include a volatile liquid within the vessel 104, similar to as shown in FIG. 3A. Similar to the air freshener 100, the air freshener 500 may be inverted (e.g., with its bottom end 105 facing upward), causing the volatile liquid within the vessel 104 to be forced against the first wick 108 by gravity and to evaporate therethrough into the space 134 between the first wick 108 and second wick 128.

The air freshener 500 of FIG. 5 may be beneficial, for example, in a situation where the user wishes to merely place the air freshener 500 on a surface (e.g., by placing the drip collection member 130 directly on a surface) instead of mounting a separate holder to a wall. That said, a design like the design shown in FIG. 5 may also be used with a holder (such as, for example, a holder similar to holder 122 of FIG. 3B, but with the support members 132 and drip collection member 130 removed therefrom). Such a design could be beneficial, for example, such as to enable easy replacement of the second wick 128 every time the fragrance container (and thus the first wick 108) is replaced.

### Additional Wick Examples

Turning now to FIGS. 6A-6C, these figures are schematic top views of a fragrance container that can be used with various embodiments disclosed herein. For example, FIG. 6A is a schematic top view of the lid 106 of the container 102 of FIG. 1D, showing the wick 108 positioned within the opening 109 of the lid 106. FIGS. 6B and 6C illustrate an alternative embodiment that includes four openings 609 in the lid 606 instead of a single opening. Such a design can be used to, for example, tune or adjust the amount of exposed wick area to a particular chemical formulation’s volatility and/or to a particular wicking material density, in order to maintain relatively long usability of the air freshener even for higher volatility fragrance blends. For example, for a chemical formulation that has a relatively low vapor pressure, all four of the openings 609 may have wicks 608 exposed therethrough. In another example, such as for use with a chemical formulation that has a relatively high vapor pressure, some of the openings 609 may be closed off, such as by positioning plugs 611 therein, leading to a smaller amount of exposed surface area of wicks 608.

It should be noted that the configurations shown in FIGS. 6B and 6C may be accomplished in various ways. For example, each of the four openings 609 may have an individual wick 608 positioned therein in the version of FIG. 6B. As another example, a single wick (such as wick 108 of FIG. 6A) may be positioned within the lid 606, and just exposed through the four openings 609 (in FIG. 6B) or through two of the openings 609 (in the version of FIG. 6C). It should also be noted that various other configurations may be used, including configurations that utilize less or more wicks or openings, configurations that utilize differently sized or shaped openings, and/or the like.

### Additional Example Air Freshener

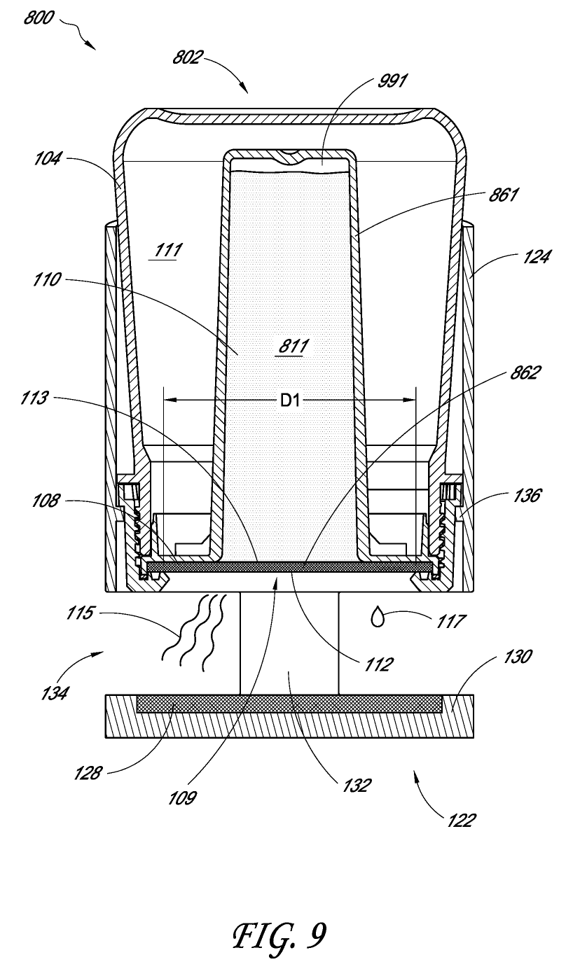

FIG. 8A-10C illustrate another embodiment of an air freshener 800, and various components thereof. More specifically, FIGS. 8A-8E depict various views of a container 802 of the air freshener 800, FIG. 9 illustrates a cross-sectional view of the air freshener 800 including the container 802 held in an inverted orientation by the holder 122, and FIGS. 10A, 10B, and 10C are perspective views of a reservoir body 861, vessel 104, and lid 106 of the container 802, respectively. The air freshener 800 has many similarities to other air fresheners disclosed herein, including air fresheners 100 and 500 described above, and the same or similar reference numbers are used to refer to the same or similar components. Any of the other air fresheners disclosed herein, including but not limited to air fresheners 100 and 500, can be modified to include one or more of the features described below with reference to the air freshener 800. Likewise, the air freshener 800 can be modified to include one or more of the features described elsewhere herein with reference to other air fresheners, including but not limited to air fresheners 100 and 500. For conciseness, the descriptions provided below focus primarily on differences in air freshener 800 as compared to other air fresheners disclosed herein, and descriptions provided elsewhere herein with respect to other air fresheners, such as air fresheners 100 and 500, are incorporated by reference herein.

With reference to FIG. 8A, this figure is a perspective view of the container 802, which shares several similarities to the container 102 shown in, for example, FIG. 1E. For example, the container 802 comprises a vessel 104 and a lid 106 attached thereto. The main difference from container 102 that is visible in this exterior view is that the lid 106 of container 802 includes a different cover 816 than the cover 116 of FIG. 1E. Similar to the cover 116, the cover 816 is desirably formed as a separable part of the lid 106 (e.g., separable from a main body 853 of the lid at seam 851), and includes a pull tab or ring 118 that makes it easier for a user to separate the cover 816 from the main body 853. The cover 816, however, includes a depression 855 that is not seen in the cover 116 of FIG. 1E.

In this embodiment, the depression 855 is a concave depression that generally corresponds to a convex inwardly protruding portion 860 on the underside of the cover 816, as described below with reference to FIG. 8C. Having such a depression 855 is not necessarily a requirement, however, and other versions of the cover 816 may include one or more inwardly protruding portions on the underside of the cover 816 without having a corresponding depression on the top side of the cover 816. Having a corresponding depression 855 on the top side of the cover 816 can be beneficial, however, such as to increase manufacturability of the lid 106, reduce material waste, and/or the like.

Turning to FIG. 8C, this figure illustrates a cross-sectional view of the container 802, taken through the section plane indicated on the top view of FIG. 8B. FIG. 8C shows that the container 802 includes additional features that are the same as or have similarities to the container 102 described above. For example, the container 802 includes a first wick 108 that can be the same as or similar to the first wick 108 of container 102, and the container 802 includes a volatile liquid 110 that is in contact with an underside of the first wick 108. One difference, however, is that the cavity 111 of vessel 104 is no longer where the volatile liquid 110 is positioned. Instead, in this embodiment, a separate reservoir body 861 (alternatively referred to as an insert, airless insert, and/or the like) is inserted into the cavity or chamber 111 of the vessel 104, and the volatile liquid 110 is positioned within a separate cavity or chamber 811 of the reservoir body 861. Accordingly, the cavity 811 of the reservoir body 861 acts as a liquid chamber, and the cavity 111 of the vessel 104 acts as an air chamber that is not in fluid communication with the cavity 811 of the reservoir body 861.

In general, the volume of the liquid chamber or cavity 811 is defined by annular wall 893 of the reservoir body 861, base or floor 894 of the reservoir body 861, and the inner surface of the first wick 108 that covers an opening 1073 (see FIG. 10A) into the cavity 811. Additionally, the volume of the air chamber or cavity 111 is generally defined by annular wall 891 of the vessel 104, base or floor 892 of the vessel 104, and the surfaces of the reservoir body 861 that are in contact with the air chamber 111 (e.g., wall 893, base or floor 894, ledge 895, and annular rim 897).

Desirably, the liquid chamber 811 can be completely full of the volatile liquid 110, with no air in the liquid chamber 811, in the “ready to activate” state shown in FIG. 8C (e.g., the state that still has the cover 816 installed in the closed position that seals the first wick 108 from the external environment). Although having the liquid chamber 811 completely full of the volatile liquid 110 is desirable, it is not a requirement, and some embodiments may have some airspace within the liquid chamber 811 prior to removal of the cover 816. Having the liquid chamber 811 completely full of the volatile liquid 110, with no airspace in the liquid chamber 811, can be desirable, however, to help reduce the effects of changes in environmental barometric pressure on the operation of the air freshener.

As a hypothetical example, if the air freshener were delivered in the “ready to activate” state with the liquid cavity 811 being filled with 75% by volume volatile liquid 110 and 25% by volume air, such air freshener may experience greater variability in evaporation of the volatile liquid through the wick over time as a result of external environment barometric pressure changes than an air freshener that is delivered with the liquid cavity 811 being filled 100% by volume, or close to 100% (such as at least 90%, 95%, 96%, 97%, 98%, or 99%) with the volatile liquid 110. It should be noted that, over time, even with an air freshener like shown in FIG. 8C that has the liquid cavity 811 fully filled with volatile liquid 110 in the “ready to activate” state, air will eventually diffuse through the first wick 108 during use as the volatile liquid 110 evaporates into the environment, and at some point—such as when the liquid cavity 811 becomes 75% by volume liquid and 25% by volume air—the air freshener may operate similarly to the hypothetical air freshener described above that started with such a configuration. The time up until that point, however, will desirably have more consistent and/or less variable output.

One benefit of having the liquid chamber 811 be within a separate reservoir body or insert 861 is that the size or volume of the cavity 811 can be tailored to the expected evaporation rates of different volatile liquids 110. For example, if a first volatile liquid is expected to evaporate at twice the speed of a second volatile liquid, then the reservoir body 861 for the first volatile liquid may be designed to create a liquid cavity 811 that is approximately twice the volume of the liquid cavity of the reservoir body design for the second volatile liquid. By making these changes in volume to the reservoir body 861, instead of to the vessel 104, the same vessel 104 may be used for a variety of different volatile liquids or fragrances, and the external appearance of the fragrance container 802 may be the same regardless of changes to volume in the liquid cavity 811 for different volatile liquids. Additionally, even when using the same volatile liquid, different reservoir bodies 861 may be desirable to use for different intended useful lives of the air freshener. For example, some users may prefer an air freshener that has a larger liquid cavity and lasts for a longer time, while other users may prefer a lower-cost air freshener that has a smaller liquid capacity and lasts for a shorter time. In some embodiments, the volume of the liquid chamber 811 is at least 25% of the volume of the air chamber 111. In some embodiments, the volume of the liquid chamber 811 is at least 20%, 30%, 40%, 50%, 60%, 70%, 80%, 90%, 100%, 125%, 150%, or 200% of the volume of the air chamber 111. In some embodiments, the volume of the liquid chamber 811 is within a range of 25%-75%, 50%-100%, 75%-125%, 25%-125%, or 100%-200% of the volume of the air chamber 111. To avoid potential confusion, each of the percentages in the three preceding sentences is referring to a comparison between the volume of the liquid chamber and the volume of the air chamber, with each of those two volumes being separate non-overlapping volumes. In other words, the volume of the liquid chamber and the volume of the air chamber are defined as described above (e.g., the volume of the liquid chamber is defined by annular wall 893, base 894, and the inner surface of the first wick 108, and the volume of the air chamber is defined by annular wall 891, base 892, and the surfaces of the reservoir body 861 that are in contact with the air chamber). In general, to obtain the longest life within the smallest package, while still maintaining the benefits of the air chamber, the volume of the liquid chamber can be increased to a relatively large value with respect to the volume of the air chamber, but desirably not so far that any portion of the reservoir body wall 893 can potentially contact the vessel wall 891 as a result of user handling, thermal expansion and contraction, expansion and contraction due to pressure changes, and/or the like.

Another benefit of having the liquid chamber 811 be within a separate reservoir body or insert 861 is that the reservoir body features that mostly or at least partially define the volume of the liquid chamber 811 (e.g., annular wall 893 and base or floor 894) are desirably hidden from and/or inaccessible from the external environment. Accordingly, handling of the container 802 by a user, which could include, for example, inadvertent or intentional squeezing of the wall 891 of the vessel 104, will desirably have little or no effect on the volume of the liquid chamber 811 and/or the pressure within the liquid chamber 811. Stated another way, in an embodiment that places the volatile liquid 110 directly within the chamber 111 of the vessel 104 (such as the embodiment shown in FIG. 3A) handling of the vessel 104 by a user (such as inadvertent or intentional squeezing of the wall 891) may result in pressure changes within the chamber 111 that cause some of the volatile liquid 110 to be forced through the wick 108 in liquid form, thus leaking some of the liquid into the external environment. In an embodiment such as shown in FIG. 8C, however, even if the wall 891 of the vessel 104 is squeezed inward, this will desirably have little or no effect on the pressure within liquid cavity 811, particularly if the air chamber 111 is vented to the external environment (as further described below with reference to FIGS. 8D and 8E). It should be noted, however, that having an air chamber 111 and a liquid chamber 811 arrangement as shown in FIG. 8C may even provide some benefit if the air chamber 111 is not vented to the environment (such as reduced leakage over a version where one would be directly squeezing the liquid chamber wall), and thus having the air chamber 111 vented to the environment is not necessarily a requirement in all embodiments.

With continued reference to FIG. 8C, this figure also illustrates another feature that can help to prevent liquid leaks or drops, particularly when first activating the air freshener. Specifically, the first wick 108 desirably comprises a relaxed shape or state that is generally a flat disc shape (similar to the first wick 108 of air freshener 100). FIG. 9, which shows the air freshener 800 in use—with the cover 816 removed from the container 802—shows the flat or relaxed state or shape of the first wick 108. FIG. 8C, however, which shows the state of the container 802 prior to activation, shows that the first wick 108 is in a deformed state, configuration, or shape. Specifically, an inwardly protruding portion 860 of the cover 816 is pressed against a middle or inner portion 862 of the first wick 108 and causing the middle or inner portion 862 of the first wick 108 to deform downward or inwardly (e.g., in a direction that tends to reduce the volume of the liquid chamber 811). When the air freshener is activated, such as by removing the cover 816 and inverting the container 802, the inwardly deformed portion 862 of the wick 108 will no longer be forced inward by the cover 816, and thus the wick 108 will tend to return to its relaxed or flat state. Such change in the wick 108 will tend to increase the volume of the liquid cavity 811, thus creating a negative pressure differential within the liquid cavity 811 with respect to the external environment. This will tend to cause liquid that was at the external or exposed surface of the first wick 108 to be drawn back through the wick 108 and/or into the liquid cavity 811, potentially along with at least some air from the external environment, in order to equalize the pressure between the liquid cavity 811 and the external environment. One benefit of this is that this will tend to reduce or eliminate initial liquid drips from the wick 108 upon initial activation of the air freshener.

As mentioned above, the cover 816 desirably comprises an exterior depression 855 that generally corresponds to the inwardly protruding portion 860 of the cover 816 (e.g., the exterior depression 855 may comprise a radius that is smaller than the radius of the inwardly protruding portion 860 but with both radiuses having the same vertex). This can be desirable, for example, for the cover 816 to maintain a relatively consistent material thickness, to reduce material waste, to reduce weight, to make manufacturing easier, and/or the like. Such a feature is not required, however, and some embodiments may include an inwardly protruding portion 860 without a corresponding external depression 855 and/or with a differently shaped external depression 855. Further, although this embodiment includes a single convex inwardly protruding portion 860 that encompasses a majority of the diameter of the opening 1073 into liquid chamber 811 (see FIG. 10A), other embodiments may vary the size of the protrusion or protruding portion 860, may include more than one protrusion or protruding portion 860, and/or the like. For example, in this embodiment, a diameter D5 of the inwardly protruding portion 860 is desirably at least 80% of a diameter D6 of the opening 1073. In some embodiments, the diameter D5 is desirably at least 50%, 60%, 70%, 80%, or 90% of diameter D6. As shown in FIG. 8C, in this embodiment, the diameter D6 of opening 1073 is defined as the diameter to the outer edge of the corner radius at the upper end of the wall 893. The same percentages discussed above may alternatively be applied to a diameter measurement of the opening at the inner edge of such a corner radius or chamfer.

In general, the larger the diameter D5, the more the volume of the liquid cavity 811 can be reduced when the wick 108 is forced into the deformed state, and thus the greater the pressure differential will be when the cover 816 is removed. Further, the larger the diameter D5, the larger the radius of the convex protrusion or protruding portion 860 may be. This can be desirable, for example, because the larger the radius is, the less stress will be put on the wicking material of the first wick 108. As discussed elsewhere herein, the first wick 108 desirably comprises a pulp-based wicking material, and such material may tend to deteriorate or take on a permanent deformation if the radius of the inwardly protruding convex portion 860 is too small. In some embodiments, the radius R1 of the inwardly protruding portion 860 is at least 1.5 times the outer diameter D2 of the first wick 108 (see dimension D2 of FIG. 3A). In some embodiments, the radius R1 is at least 1.0, 1.5, 1.75, 2.0, 2.25, 2.5, 2.75, or 3.0 times the outer diameter D2 of the first wick 108.

Another way to limit permanent deformation of the wick, while still having a sufficient amount of liquid chamber volume change between the deformed and relaxed states of the wick, is to have diameter D5 be relatively close to diameter D6 (such as diameter D5 being at least 80% or 90% of diameter D6), while limiting the amount of axial deformation of the deformed portion of the wick 862 (represented by, e.g., the axial distance Z1 shown in FIG. 8C, which is measured along the axial direction from the underside of the portion of the cover 816 that engages the outer undeformed portion 864 of the wick to the underside of the inwardly protruding portion 860 of the cover). In other words, the larger diameter D5 is, the smaller Z1 needs to be to result in the same amount of liquid chamber volume change—and making Z1 smaller will generally result in less stress being introduced into the wick than if Z1 were larger. For example, D5 may desirably be at least 80% or 90% of D6, with D5 also being within a range of 5-15 times Z1. In some embodiments, D5 may desirably be at least 80% or 90% of D6, with D5 also being within a range of 5-10, 7-15, or 5-20, or 10-25 times Z1.

With continued reference to FIG. 8C, it can be seen that the first wick 108 also includes an outer annular portion 864 that is radially outward of the deformed portion 862 and that remains generally flat (e.g., does not move along the axial direction) when the middle portion 862 is deformed inward. The lower surface of the outer portion 864 of the first wick 108 is desirably placed upon and supported by upper surface 1071 of annular top portion 1065 of the reservoir body 861 (see FIG. 10A). The upper surface of the outer portion 864 of the first wick 108 is desirably held in place by the lid 106, and more specifically by the protrusion, tooth, annular rim, and/or the like 889 shown in the detail cross-sectional view of FIG. 8E. Desirably, the protrusion or tooth 889 of the lid 106 can at least partially bite into (e.g., be compressed or forced into) the wick 108 in order to resist radially inward movement of the outer portion 864 of the wick 108 when the middle portion 862 of the wick 108 is deformed inwardly by the cover 816. In some embodiments, the space or cavity 887 next to the protrusion or tooth 889 (see FIG. 8E) can allow for the protrusion or tooth 889 to at least partially compress the material of the wick 108, with at least some adjacent material of the wick 108 being displaced upward into the cavity 887. This can further help to maintain the outer portion 864 of the wick 108 in place when the middle portion 862 of the wick 108 is deformed downward (and thus pulling radially inward on the outer portion 864 of the wick 108). In some embodiments, the size of the opening into the liquid chamber 811 (e.g., diameter D6 of FIG. 8C) is substantially smaller than the size of the wick (e.g., diameter D2 of FIG. 8C), such as to increase the amount of undeformed portion 864 of the wick, to further reduce the chances of damaging the wick 108 when the inner portion 862 is deformed. For example, the size of the opening may in some embodiments be no more than 50%, 60%, 70%, or 80% of the size of the wick. It should further be noted that the deformation of the middle portion 862 of the wick 108 toward the liquid chamber 811 in a manner that reduces the volume of the liquid chamber may not be the only feature that helps to reduce or eliminate initial leaks or drips from the wick 108 when the air freshener is initially activated (e.g., when the cover 816 is removed and the container 802 is inverted). For example, at least some portions of the wick 108 may be compressed (e.g., compressed along the axial direction and/or with respect to the thickness of the sheet material of the wick) by the cover 816 when the cover 816 is in the closed configuration. Removal of the cover 816 may allow those portions of the wick to decompress (e.g., to expand along the axial direction and/or with respect to the thickness of the sheet material of the wick), which may cause some liquid that may have been present on the exterior surface of the wick 108 to be drawn back into the wick 108. For example, there is an annular region of the outer portion 864 of the wick that will desirably be compressed between the flat underside surface of the cover 816 (see surface 863 of FIG. 10C) and the flat upper surface of the reservoir body 861 (see surface 1071 of FIG. 10A) when the cover 816 is in its closed configuration as shown in FIG. 8C. More specifically, the annular region of the wick that is compressed between those two surfaces may be defined as the annular region between the edge of the opening into the liquid chamber (e.g., diameter D6) and the edge of the flat underside of the cover 816 (e.g., diameter D7). In some embodiments, the annular region that is compressed between the cover 816 and reservoir body 861 is defined as the region between diameters D6 and D7, with a ratio of diameter D6 to diameter D7 being within a range of 0.6 to 0.8. In some embodiments, the annular region that is compressed between the cover 816 and reservoir body 861 is defined as the region between diameters D6 and D7, with the ratio of diameter D6 to diameter D7 being within a range of 0.4 to 0.6, 0.7 to 0.9, or 0.5 to 0.9. Further, although in this embodiment the inwardly protruding portion 860 of the cover 816 does not extend radially beyond the opening into the liquid chamber (e.g., diameter D5 is not larger than diameter D6), some embodiments may extend the inwardly protruding portion 860 of the cover 816 radially beyond the opening into the liquid chamber (e.g., diameter D5 may be larger than the diameter D6). This may, for example, result in some compression of the wick 108 between the inwardly protruding portion 860 and the reservoir body. This may also, for example, result in a more gradual deformation of the wick 108, further reducing the chances of damaging the wick 108. In some embodiments, a ratio of diameter D5 to diameter D7 is within a range of 0.5 to 0.7. In some embodiments, the ratio of diameter D5 to diameter D7 is within a range of 0.6 to 0.8, 0.7 to 0.9, or 0.7 to 1.0. In some embodiments, the size of the inwardly protruding portion 860 may be considered as a ratio of the size of the wick 108 (e.g., diameter D2). For example, in some embodiments, a ratio of diameter D5 to diameter D2 is within a range of 0.4 to 0.5. In some embodiments, the ratio of diameter D5 to diameter D2 is within a range of 0.4 to 0.6, 0.5 to 0.7, or 0.6 to 0.9. In some embodiments, the size of the inwardly protruding portion 860 may be considered as a ratio of the size of the opening into the liquid chamber (e.g., diameter D6). For example, in some embodiments, a ratio of diameter D5 to diameter D6 is within a range of 0.75 to 1.00. In some embodiments, the ratio of diameter D5 to diameter D6 is within a range of 0.8 to 1.0, 0.9 to 1.1, 1.0 to 1.5, or 1.25 to 1.60.

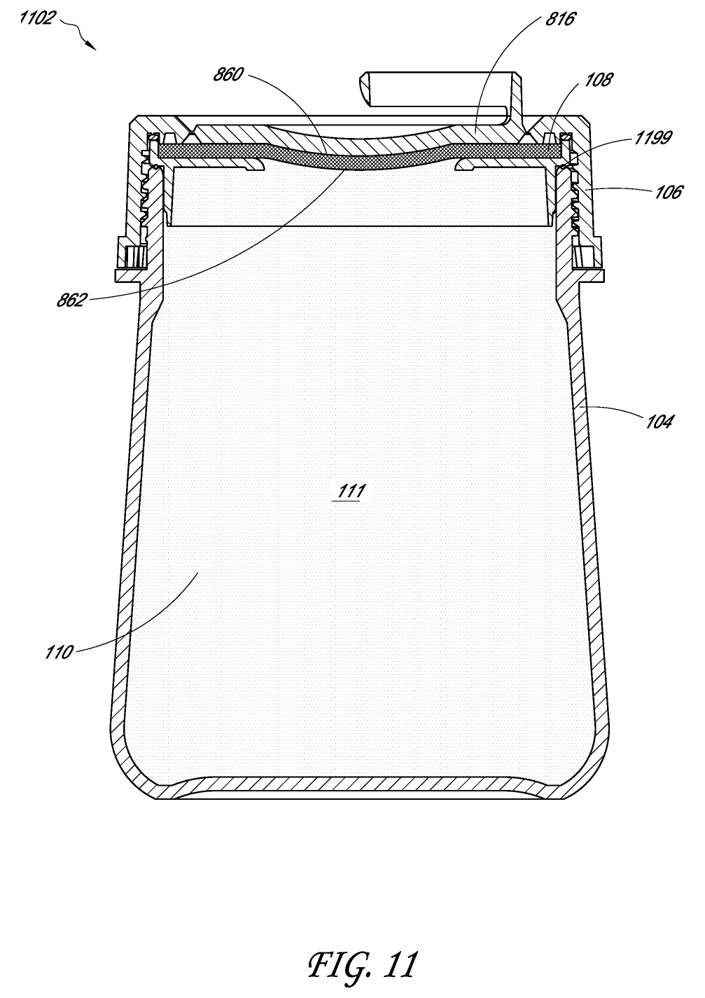

It should be noted that the features described above with respect to deforming a portion of the wick 108 inward and/or compressing a portion of the wick 108 when the cover 816 is in the closed configuration are not limited to air fresheners that have a separate liquid chamber 811 positioned within an air chamber 111 (as shown in FIG. 8C). For example, FIG. 11 illustrates an alternative embodiment of an air freshener container 1102 that is similar to air freshener container 802 (and can be used with the same holder 122) except for the following differences. First, the wall 893 and base 894 have been removed, in order to have a single chamber 111 that acts as the liquid chamber, without there being a separate air chamber. Second, the chamber 111 is no longer vented to the external environment. Instead, an O-ring 1199 has been added that seals the chamber 111 from the external environment, except for through the first wick 108 (once the cover 816 has been removed). Such a configuration can be used to, for example, obtain the wick deformation benefits of reducing initial drips or leaks at initial activation, while allowing use of the full volume of the vessel 104 to store volatile liquid. In such a configuration, the vessel 104 may optionally be referred to as a reservoir body, since the vessel—instead of a separate reservoir body—at least partially defines the liquid chamber.

Turning now to FIGS. 8D and 8E, these figures illustrate additional details of how the air chamber 111 can be vented to the external environment. In this embodiment, the air chamber 111 is desirably vented to the external environment between the vessel 104 and the lid 106, such as through a vent path or flow path 883 depicted in the detail view of FIG. 8E. More specifically, the vent path 883 passes from the cavity 111, through a space 1063 between the reservoir body 861 and the vessel 104, through a space between the vessel 104 and main body 853 of the lid 106 (e.g., through the space that includes threads 807a of the lid and 807b of the vessel), through a space between mating teeth 1081 of the lid and 1083 of the vessel, and to the external environment. As can be seen in FIG. 8E, an O-ring 885 is positioned between the main body 853 of the lid and a lip 1069 of the reservoir body 861, thus sealing the first wick 108 from the vent path 883 (at least while the cover 816 is still installed/intact).

Desirably, the reservoir body 861 is shaped to facilitate the vent path 883 by forming the space 1063. For example, with reference to FIG. 10A, which is a perspective view of the reservoir body 861, the reservoir body 861 comprises an annular top portion 1065 that includes an annular rim 897 having a plurality of protrusions 1062 extending radially therefrom. Further, there are a plurality of spaces 1063 formed between the protrusions 1062. The protrusions 1062 desirably help to position and/or center the reservoir body 861 with respect to the upper neck 898 of the vessel 104 (see FIG. 8C). The cross-sectional view of FIG. 8C is taken through a section plane that passes through protrusions 1062, and thus it can be seen in FIG. 8C that the protrusions 1062 are engaged with or positioned close to the inner surface of the neck 898. The cross-sectional view of FIG. 8D, however, is taken through a section plane that passes through the space 1063 between two protrusions 1062, and thus it can be seen in FIG. 8D (and more clearly in detail view FIG. 8E) that the vent path 883 can pass through the space 1063.

It should be noted that the vent path 883 shown in FIG. 8B is merely one example of how the air chamber 111 can be vented to the atmosphere, and various other venting techniques may be utilized (alternatively or in combination). For example, in some embodiments, one or more holes, openings, apertures, and/or the like may be included in or through the wall 891 and/or base 892 of the vessel 104. For example, the dashed lines in FIG. 8D identified with element number 881 show one example of an area where a hole may be positioned through the base 892. Various embodiments may include more than one such hole, may position the hole or holes through the base 892 and/or the wall 891, and/or the like. Further, some embodiments may include a wall that is positioned radially outward from the reservoir body wall 893 and that forms an annular air space between the wall 893 and the wall that is radially outward of the wall 893, but without forming an air chamber 111. For example, some embodiments may include a structure that generally corresponds to the wall 891 of the vessel 104, but without including the base 892. The structure may be the same or similar generally tapered shape as wall 891, or may be another shape, such as a cylinder, a square, a tapered shape that tapers in the other direction, and/or the like. Such a configuration (e.g., a configuration that creates air space in an annular area radially between wall 893 and another structure radially outward of wall 893, but without necessarily defining an air “chamber”) may still be beneficial, for example, such as to avoid a user directly handling the reservoir body 861. In any of these alternative designs, and in the design shown in FIG. 8D, it can be desirable for at least a majority of the air present within the air chamber 111 (or within the bounds of the wall that is positioned radially outward from the reservoir body wall 893, in a version that does not necessarily create an air “chamber”) to be positioned within an annular space between the first wall (e.g., wall 83) and the second wall (e.g., wall 891). For example, with reference to FIG. 8D, element number 875 represents such an annular space, with the arrows above and below element number 875 indicating the height of the annular space 875. In some embodiments, at least 75% of the air present within air chamber 111 (or within the bounds of the wall that is positioned radially outward from the reservoir body wall 893, in a version that does not necessarily create an air “chamber”) is within the annular space 875. In some embodiments, at least 50%, 60%, 70%, 80%, or 90% of the air present within air chamber 111 (or within the bounds of the wall that is positioned radially outward from the reservoir body wall 893, in a version that does not necessarily create an air “chamber”) is within the annular space 875. Having a majority of more of the air within the annular space 875 can be beneficial, for example, to help maximize the size of the liquid chamber while keeping a relatively significantly sized buffer or airspace between the liquid chamber wall and the outer air chamber wall.

Turning to FIG. 9, this figure shows a cross-sectional view of the air freshener 800 in use, with the container 802 inverted and installed into the holder 122 (which may be the same as or similar to the holder 122 of FIG. 3C), and with the cover 816 having been removed from the container 802. As can be seen in this figure, the first wick 108 has returned to its relaxed or flat state or configuration (e.g., the inner or middle portion 862 of the wick 108 is no longer deformed inward). The air freshener 800 will operate similarly to as described above with reference to FIG. 3C, and the same or similar reference numbers are used to refer to the same or similar features.

It can be seen in FIG. 9 that some airspace 991 has formed within liquid chamber 811. For example, as described above, when the cover 816 is removed and allows the first wick 108 to return to its relaxed state, desirably some air will diffuse from the external environment through the first wick 108 and into the liquid chamber 811. This air will then move to the upper end of the liquid chamber 811 into airspace 991. Shortly after activation of the air freshener 800 (e.g., shortly after removing cover 816), the size or volume of the airspace 991 may be smaller than, close to, or generally equivalent to the difference in volume of the liquid chamber 811 between when the cover 816 is installed (with the first wick 108 in its deformed state) and the cover 816 is not installed (with the first wick 108 in its relaxed state). Over time, as the volatile liquid 110 evaporates into the external environment through the first wick 108, additional air will enter the liquid chamber 811, thus making the airspace 991 increase in size.

As discussed above, one of the benefits of using the reservoir body 861 to create a liquid chamber 811 that is separate from the chamber 111 defined by the vessel 104 is that the size of the liquid chamber 811 can be tuned to the expected evaporation rate for each of a variety of volatile liquids 110. Accordingly, with a design as shown in FIG. 9, various volatile liquids 110 may be used with various sized liquid cavities 811, which each start out with only a relatively small airspace 991 in the liquid cavity 811 after removal of the cover 816. In a design that places the volatile liquid 110 within chamber 111 formed by the vessel 104, on the other hand (such as, for example, air freshener 100), different sizes of airspace may be present within the chamber 111 at activation time (assuming the chamber 111 is the same size for each design, but different amounts of starting volatile liquid are included, depending on each formulation’s expected rate of evaporation), which may lead to more variation in fragrance output responsive to external environment pressure changes, particularly in versions that have a larger initial airspace than others.

Turning to FIGS. 10B and 10C, these figures show additional detail of how the lid 106 is desirably locked onto the vessel 104 at manufacturing time, thus reducing the risk that an end user accidentally removes the lid and spills the volatile liquid. Specifically, FIG. 10B shows that the vessel 104 includes a plurality of teeth 1083 positioned adjacent thread 807b. Likewise, FIG. 10C shows that the lid 106 includes a plurality of teeth 1081 positioned adjacent thread 807a. When the product is assembled, the thread 807a of the lid 106 is engaged with thread 807b of the vessel 104, and as the lid 106 is turned onto the vessel 104 the teeth 1081 will engage teeth 1083. The teeth 1081 and 1083 are shaped such that they act as a ratchet or one-way clutch mechanism, meaning that as the lid 106 is tightened down onto the vessel 104, the teeth 1081 will rotate and advance to engage other teeth 1083 of the vessel 104, but the teeth 1083 will not allow the teeth 1081 to move the opposite direction. It should be noted that this is merely one example of how the lid 106 may be locked onto the vessel 104, and various other techniques may be used. FIG. 10C also shows that there is a flat surface or portion 863 radially outward of the inwardly protruding portion 860.

### Additional Volatile Liquid Examples

As discussed above, one of the ways various embodiments disclosed herein are able to maintain a relatively consistent output of fragrance over an extended period of time is that they may utilize a volatile liquid that is a chemical formulation or blend comprising a plurality of chemicals that each have a similar vapor pressure within a relatively small range of each other. For example, some embodiments may utilize a volatile liquid formulation that consists of a plurality of volatile chemicals, wherein the most volatile and the least volatile chemicals in the formulation have a vapor pressure difference within a 0.4mmHg range, within a 0.5mmHg range, within a 1mmHg range, within a 1.5mmHg range, within a 2mmHg range, within a 3mmHg range or any ranges between 0.4mmHg and 3mmHg. Similarly, some embodiments may utilize a volatile liquid formulation that consists of a plurality of volatile chemicals, wherein the most volatile and the least volatile chemicals in the formulation have a vapor pressure difference within an about 0.4mmHg range, within an about 0.5mmHg range, within an about 1mmHg range, within an about 1.5mmHg range, within an about 2mmHg range, within an about 3mmHg range or any ranges between about 0.4mmHg and about 3mmHg. Stated another way, for an example blend that includes a plurality of volatile chemicals all having a vapor pressure spanning within a 1mmHg range, the vapor pressures of each of the plurality of volatile chemicals forming the blend may fall within a range of, for example, 0.2-1.2mmHg, 0.5-1.5mmHg, 1.0-2.0mmHg, 3.0-4.0mmHg, 8.0-9.0mmHg, etc. In some embodiments, it may be desirable to use a blend that includes a plurality of volatile chemicals having vapor pressures within a range of, for example, 0.15-1.0mmHg, 1.5-3.0mmHg, or 0.1-0.45mmHg.

In some embodiments, the relatively narrow ranges of vapor pressures described above may apply to every chemical that is included in the volatile liquid blend. In some embodiments, however, these relatively narrow ranges of vapor pressures may apply to a subset of chemicals that are included in the volatile liquid blend. For example, some volatile liquid blends may include one or more chemicals that form a relatively small percentage by volume of the total composition of the blend and have a vapor pressure that is outside of the above referenced ranges. For example, some volatile liquid blends may include a plurality of chemicals that form at least 90% by volume of the volatile liquid blend, with the above referenced ranges of vapor pressure applying only to that plurality of chemicals. In some embodiments, some volatile liquid blends may include a plurality of chemicals that form at least a majority, 60%, 70%, 75%, 80%, 85%, 90%, 95%, or 99% by volume of the volatile liquid blend, with the above referenced ranges of vapor pressure differences applying only to that plurality of chemicals.

As discussed above, it can be desirable to keep the vapor pressures of the plurality of chemicals that form the volatile liquid blend within a relatively narrow range, which, for example, can help to avoid clogging of the wick material that could otherwise significantly affect the evaporation characteristics of the volatile liquid blend over time. This benefit can be seen over a relatively wide range of vapor pressures. For example, with the example of a 1mmHg range, a first blend of chemicals having vapor pressures within a range of 0.5 to 1.5mmHg can experience this benefit, while a second blend of chemicals having vapor pressures within a range of 7.0 to 8.0mmHg can also experience this benefit. An important difference between those two blends, however, is that, assuming the same wick and wick exposure area are used, the second blend of chemicals having a higher average vapor pressure than the first blend of chemicals will tend to evaporate through the wick at a faster rate than the first blend of chemicals, and thus the output of the air freshener will be shorter for a same volume of starting chemical.

In view of the above, in some embodiments, it may be desirable to adjust or tune the wick exposure area to the average and/or median vapor pressure of a particular chemical blend. For example, one embodiment of an air freshener may utilize a circular wick exposure having a diameter of 55mm (e.g., diameter D1 of FIGS. 1D, 7, or 9), which corresponds to an exposure area of approximately 2376mm². If that embodiment were used with a chemical blend having an average vapor pressure of 1 mmHg, then a ratio of exposure area to the average vapor pressure would be. As another example, if that embodiment were used with a chemical blend having an average vapor pressure of 0.5 mmHg, then the ratio of exposure area to the average vapor pressure would be 4752. As another example, if that embodiment were used with a chemical blend having an average vapor pressure of 1.5 mmHg, then the ratio of exposure area to the average vapor pressure would be 1584. In some embodiments, it may be desirable to maintain the ratio of exposure area to average vapor pressure to within a range of 1500 to 5000 (or about 1500 to about 5000), with the exposure area measured in square millimeters and the average vapor pressure measured in millimeters of mercury. In some embodiments, it may be desirable to maintain the ratio within a smaller range, such as 1500 to 4000 (or about 1500 to about 4000), 1500 to 3000 (or about 1500 to about 3000), 2000 to 3000 (or about 2000 to about 3000), 2500 to 5000 (or about 2500 to about 5000), and/or the like. In some embodiments, particularly in cases of relatively low vapor pressures (such as, for example, a range of about 0.1-0.45mmHg with a median vapor pressure of about 0.28mmHg), it may be desirable to use an even higher ratio, such as within a range of 10,000 to 20,000 (or about 10,000 to about 20,000).

In some embodiments, as the average or median vapor pressure increases, it may be desirable to utilize a smaller exposure area and a smaller ratio of exposure area to average vapor pressure. For example, with a chemical blend having a much higher average vapor pressure of 3.0mmHg, it may be desirable to use a much smaller wick, such as a wick having an exposure diameter of 12.7mm (or about 12.7mm) (e.g., exposure area of 127mm² (or about 127mm²)), leading to a ratio of 42 (or about 42). In some embodiments, it may be desirable for the ratio to be within a range of 30 to 50, 20 to 60, 10 to 70, and/or the like. In some embodiments, it may be desirable for the ratio to be within a range of about 30 to about 50, about 20 to about 60, about 10 to about 70, and/or the like. As another example, with a chemical blend having an average vapor pressure of 1.5 mmHg, it may be desirable to use a wick having exposure diameter between 12.7mm and 55mm (or about 12.7mm and about 55mm), such as 25mm (or about 25mm) (e.g., exposure area of 491mm² (or about 491mm²)), leading to a ratio of 327 (or about 327). In some embodiments, it may be desirable for the ratio to be within a range of 300 to 350, 250 to 400, 200 to 450, and/or the like. In some embodiments, it may be desirable for the ratio to be within a range of about 300 to about 350, about 250 to about 400, about 200 to about 450, and/or the like. It should be noted that these numbers are merely examples and are not intended to limit the disclosure.

As additional examples, it has been found through testing that it can be desirable to use a wick with an exposure diameter of 20mm (or about 20mm) (e.g., exposure area of 314mm² (or about 314mm²)), 50mm (or about 50mm) (e.g., exposure area of 1,964mm² (or about 1,964mm²)), or 75mm (or about 75mm) (e.g., exposure area of 4,418mm² (or about 4,418mm²)) with a chemical blend having a median vapor pressure of 2.25mmHg (or about 2.25mmHg) (e.g., a range of 1.5-3.0mmHg or about 1.5-3.0mmHg), 0.58mmHg (or about 0.58mmHg) (e.g., a range of 0.15-1.0mmHg or about 0.15-1.0mmHg), or 0.28mmHg (or about 0.28mmHg) (e.g., a range of 0.1-0.45mmHg or about 0.1-0.45mmHg), respectively. These examples correspond to ratios of exposure area to median vapor pressure of 140, 3,386, and 15,779 (or about 140, 3,386, and 15,779), respectively.

In some embodiments, as the average vapor pressure of the volatile liquid blend intended to be used with the air freshener increases, other adjustments or tweaks (in lieu of or in addition to changing the ratio of exposure area to average vapor pressure) may be made. For example, a larger starting volume of liquid may be used and/or a wick material with higher resistance to flow therethrough may be used.

As used herein, the term “average vapor pressure” is intended to refer to a weighted average of the vapor pressures of the plurality of chemicals that form a volatile liquid blend, and the term “median vapor pressure” is intended to refer to the vapor pressure value that is halfway between the vapor pressure of the least volatile chemical in the blend and the vapor pressure of the most volatile chemical in the blend. For example, if all of the chemicals that form a volatile liquid blend are being referenced, then the average vapor pressure would be a weighted average of the vapor pressure of each of the individual chemicals, weighted by the relative volumes of each of the chemicals, and the median vapor pressure would be halfway between the vapor pressure of the least volatile chemical in the blend and the vapor pressure of the most volatile chemical in the blend. As another example, if a subset of the chemicals that form a volatile liquid blend are being referenced (for example, when referencing that at least 90% by volume of the chemicals that form a chemical blend have vapor pressures within a particular range), then the average vapor pressure would be a weighted average of the vapor pressures of each of the chemicals of that subset of the chemicals, weighted by the relative volumes of each of the chemicals within that subset, and the median vapor pressure would be halfway between the vapor pressure of the least volatile chemical in the subset and the vapor pressure of the most volatile chemical in the subset.

### Additional Information

From the foregoing description, it will be appreciated that embodiments of an inventive air freshener are disclosed. While several components, techniques and aspects have been described with a certain degree of particularity, it is manifest that many changes can be made in the specific designs, constructions and methodology herein above described without departing from the spirit and scope of this disclosure.

Certain features that are described in this disclosure in the context of separate implementations and/or “some embodiments” can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as any subcombination or variation of any subcombination.

Moreover, while methods may be depicted in the drawings or described in the specification in a particular order, such methods need not be performed in the particular order shown or in sequential order, and all methods need not be performed, to achieve desirable results. Other methods that are not depicted or described can be incorporated in the example methods and processes. For example, one or more additional methods can be performed before, after, simultaneously, or between any of the described methods. Further, the methods may be rearranged or reordered in other implementations. Also, the separation of various system components in the implementations described above should not be understood as requiring such separation in all implementations, and it should be understood that the described components and systems can generally be integrated together in a single product or packaged into multiple products. Additionally, other implementations are within the scope of this disclosure.

Conditional language, such as “can,” “could,” “might,” or “may,” unless specifically stated otherwise, or otherwise understood within the context as used, is generally intended to convey that certain embodiments include or do not include, certain features, elements, and/or steps. Thus, such conditional language is not generally intended to imply that features, elements, and/or steps are in any way required for one or more embodiments.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than or equal to 10% of, within less than or equal to 5% of, within less than or equal to 1% of, within less than or equal to 0.1% of, and within less than or equal to 0.01% of the stated amount.

Some embodiments have been described in connection with the accompanying drawings. The figures, or at least some portions of the figures, may be drawn to scale, but such scale should not be limiting, since dimensions and proportions other than what are shown are contemplated and are within the scope of the disclosed inventions. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

While a number of embodiments and variations thereof have been described in detail, other modifications and methods of using the same will be apparent to those of skill in the art. Accordingly, it should be understood that various applications, modifications, materials, and substitutions can be made of equivalents without departing from the unique and inventive disclosure herein or the scope of the claims.

What is claimed is:

1. An air freshener comprising:
a reservoir body comprising a first wall that at least partially defines a liquid chamber;
a volatile liquid positioned within the liquid chamber of the reservoir body;
a first wick that covers an opening of the reservoir body, wherein the first wick comprises a sheet of wicking material;
a vessel comprising a second wall that at least partially defines an air chamber,
wherein the first wall of the reservoir body is positioned substantially radially inward of the second wall, such that an exterior surface of the first wall is in contact with air positioned within the air chamber, and an interior surface of the first wall is in contact with the volatile liquid positioned within the liquid chamber; and
a lid coupled to a first end of the vessel and positioned such that the liquid chamber is fluidly sealed from an external environment except through the first wick,
wherein the air chamber is vented to the external environment.

2. The air freshener of claim 1, further comprising a holder that positions a second wick below and spaced apart from the first wick, with the liquid chamber positioned above the first wick.

3. The air freshener of claim 1, wherein the air chamber is vented to the external environment at least through a flow path that passes through an area between the vessel and the lid.

4. The air freshener of claim 3, further comprising a seal between the reservoir body and the lid that restricts fluid communication from an external side of the first wick to the external environment through the area between the vessel and the lid.

5. The air freshener of claim 1, wherein the air chamber is vented to the external environment through at least one opening in at least one of the second wall of the vessel or a base of the vessel.

6. The air freshener of claim 1, wherein the first wall of the reservoir body is positioned such that at least 75% of the air positioned within the air chamber is positioned within an annular space between the first wall of the reservoir body and the second wall of the vessel.

7. The air freshener of claim 1, wherein a volume of the liquid chamber is at least 25% of a volume of the air chamber.

8. The air freshener of claim 1, wherein a volume of the liquid chamber is at least 75% of a volume of the air chamber.

9. The air freshener of claim 1, further comprising a cover positioned to prevent the first wick from being exposed to the external environment until the cover is moved away from a closed position.

10. The air freshener of claim 9, wherein the cover is part of the lid.

11. The air freshener of claim 9, wherein a volume of the liquid chamber is at least partially defined by the first wick,
wherein the first wick comprises a relaxed state and a deformed state, with the deformed state corresponding to a smaller liquid chamber volume than the relaxed state, and
wherein the cover comprises an inwardly protruding portion positioned such that, with the cover in the closed position, the inwardly protruding portion of the cover forces the first wick into the deformed state.

12. The air freshener of claim 11, wherein the first wick comprises an outer portion that is compressed between the lid and the reservoir body, and an inner portion that is radially inward of the outer portion and that is positioned over the opening of the reservoir body,
wherein the inner portion of the first wick deflects along an axial direction between the deformed state and the relaxed state, and
wherein the outer portion of the first wick does not deflect along the axial direction between the deformed state and the relaxed state.

13. The air freshener of claim 1, wherein the wicking material is pulp-based wicking material.

14. An air freshener comprising:
a reservoir body comprising a first wall that at least partially defines a liquid chamber;
a volatile liquid positioned within the liquid chamber of the reservoir body;
a first wick that covers an opening of the reservoir body, such that the liquid chamber is fluidly sealed from an external environment except through the first wick,
wherein the first wick comprises a sheet of wicking material; and
a second wall positioned around and spaced apart from first wall of the reservoir body, forming an annular space between the first wall of the reservoir body and the second wall, wherein the annular space is in fluid communication with the external environment.

15. The air freshener of claim 14, further comprising a second wick spaced apart from the first wick and positioned such that, with the volatile liquid positioned above the first wick, the second wick will be below the first wick.

16. The air freshener of claim 15, further comprising a holder that supports first wick above the second wick.

17. The air freshener of claim 14, wherein the second wall is part of a vessel within which the reservoir body is substantially positioned.

18. The air freshener of claim 14, further comprising a cover positioned to prevent the first wick from being exposed to the external environment until the cover is moved away from a closed position.

19. The air freshener of claim 18, wherein the cover is part of a lid that maintains the reservoir body in a fixed position with respect to the second wall.

20. The air freshener of claim 18, wherein a volume of the liquid chamber is at least partially defined by the first wick,
wherein the first wick comprises a relaxed state and a deformed state, with the deformed state corresponding to a smaller liquid chamber volume than the relaxed state, and
wherein the cover comprises an inwardly protruding portion positioned such that, with the cover in the closed position, the inwardly protruding portion of the cover forces the first wick into the deformed state.

21. The air freshener of claim 20, wherein the first wick comprises an inner portion that is positioned over the opening of the reservoir body, and an outer portion that is positioned radially outward of the inner portion and the opening of the reservoir body,
wherein the inner portion of the first wick deflects along an axial direction between the deformed state and the relaxed state, and
wherein the outer portion of the first wick does not deflect along the axial direction between the deformed state and the relaxed state.

22. The air freshener of claim 14, wherein the wicking material is pulp-based wicking material.

23. An air freshener comprising:
a reservoir body comprising a first wall that at least partially defines a liquid chamber;
a volatile liquid positioned within the liquid chamber of the reservoir body;
a first wick that covers an opening into the liquid chamber, such that the liquid chamber is fluidly sealed from an external environment except through the first wick,
wherein the first wick comprises a sheet of wicking material; and
a cover positioned to prevent the first wick from being exposed to the external environment until the cover is moved away from a closed position,
wherein a volume of the liquid chamber is at least partially defined by the first wick,
wherein the first wick comprises a relaxed state and a deformed state, with the deformed state corresponding to a smaller liquid chamber volume than the relaxed state, and
wherein the cover comprises an inwardly protruding portion positioned such that, with the cover in the closed position, the inwardly protruding portion of the cover forces the first wick into the deformed state.

24. The air freshener of claim 23, further comprising a lid, wherein the cover is part of the lid.

25. The air freshener of claim 24, wherein the first wick comprises an outer portion that is compressed between the lid and a surface beneath the outer portion, and an inner portion that is positioned over the opening,
wherein the inner portion of the first wick deflects along an axial direction between the deformed state and the relaxed state, and
wherein the outer portion of the first wick does not deflect along the axial direction between the deformed state and the relaxed state.

26. The air freshener of claim 23, wherein the wicking material is pulp-based wicking material.

27. The air freshener of claim 23, further comprising a vessel comprising a second wall that at least partially defines an air chamber,
wherein the first wall of the reservoir body is positioned substantially radially inward of the second wall, such that an exterior surface of the first wall is in contact with air positioned within the air chamber and an interior surface of the first wall is in contact with the volatile liquid positioned within the liquid chamber, and
wherein the air chamber is vented to the external environment.

28. The air freshener of claim 27, further comprising a lid that maintains the reservoir body in a fixed position with respect to the vessel.

29. The air freshener of claim 28, wherein the cover is part of the lid.

30. The air freshener of claim 23, further comprising a holder that positions a second wick below and spaced apart from the first wick, with the liquid chamber positioned above the first wick.
